(19) United States
(12) Patent Application Publication
(10) Pub. No.: US 2025/0000992 A1
(43) Pub. Date: Jan. 2, 2025

(54) COMPOUNDS FOR THE DELIVERY OF GRANULIN ACROSS THE BLOOD BRAIN BARRIER

(71) Applicant: Eli Lilly and Company, Indianapolis, IN (US)

(72) Inventors: Alberto ALVARADO, San Diego, CA (US); Forest H. ANDREWS, Indianapolis, IN (US); David Albert DRIVER, Solana Beach, CA (US); Ross Edward FELLOWS, Westfield, IN (US); Karen Jean FRONING, Escondido, CA (US); Daniel Scott GIRARD, San Marcos, CA (US); Petra VERDINO, San Diego, CA (US); Yaming WANG, Zionsville, IN (US)

(21) Appl. No.: 18/746,732

(22) Filed: Jun. 18, 2024

Related U.S. Application Data
(60) Provisional application No. 63/509,352, filed on Jun. 21, 2023.

(51) Int. Cl. A61K 47/68; A61P 25/28

(52) U.S. Cl. CPC A61K 47/6811; A61K 47/6843; A61K 47/6889; A61P 25/28

(57) ABSTRACT

Compounds for the delivery of progranulin, a progranulin fragment, and/or at least one granulin protein subunit across the blood brain barrier. Compounds for the delivery of progranulin, a progranulin fragment, and/or at least one granulin protein subunit across the blood brain barrier, the compounds including a progranulin domain, a TfR1 binding domain, and optionally an albumin binding domain. Compounds for the delivery of wildtype or unmodified progranulin across the blood brain barrier.

### REFERENCE TO A SEQUENCE LISTING

The present application is being filed along with a Sequence Listing in ST.26 XML format. The Sequence Listing is provided as a file titled “30330” created 19 Jun. 2023 and is 66 kilobytes in size. The Sequence Listing information in the ST.26 XML format is incorporated herein by reference in its entirety.

### FIELD OF THE INVENTION

The present invention relates to novel compounds for the delivery of progranulin, or fragments thereof, across the blood brain barrier. The present invention also relates to novel compounds comprising a progranulin domain, a transferrin receptor 1 (TfR1) binding domain, and an albumin binding domain.

### BACKGROUND OF THE INVENTION

Progranulin, a 593 amino acid protein, is a precursor protein that in humans is encoded by the GRN gene. Individual granulin proteins are cleaved from progranulin. Naturally occurring progranulin includes the pro-protein, represented by para granulin (p), attached to a 7 granulin protein structure, G-F—B-A-C-D-E, where each of the granulin proteins are represented by a capital letter. In total, naturally occurring progranulin has the structure of p-G-F—B-A-C-D-E.

Various neurodegenerative disease states, such as neuronal ceroid lipofuscinosis type-11, frontotemporal dementia, Alzheimer's disease, Parkinson's disease, amyotrophic lateral sclerosis (ALS), among others, have been associated with the deficiency of progranulin in the brain or cerebrospinal fluid (CSF). Progranulin deficiency accounts for roughly 25 percent of all heritable forms of frontotemporal dementia (FTD), an early-onset neurodegenerative disease. Progranulin acts protectively in several disease models with increased progranulin levels, accelerating behavioral recovery from ischemia (Tao, J et al., (2012) Brain Res 1436, 130-136; Egashira, Y. et al., (2013). J Neuroinflammation 10, 105), suppressing locomotor deficits in a Parkinson's disease model (Van Kampen, J. M et al. (2014). PLoS One 9, e97032), attenuating pathology in a model of amyotrophic lateral sclerosis (Laird, A. S et al., (2010). PLoS One 5, e13368) and arthritis (Tang, W et al., (2011). Science 332, 478-484) and preventing memory deficits in an Alzheimer's disease model (Minami, S. S et al., (2014). Nat Med 20, 1157-1164).

Accordingly, there is a need to develop therapies that can address disorders caused by loss of progranulin function or reduced levels of progranulin. Unfortunately, it can be challenging to deliver progranulin across the Blood Brain Barrier (BBB) and/or for the provided progranulin to have a long enough half-life to persist in the brain and/or CSF in patients with a progranulin deficiency. Accordingly, there is a need for a compound that can provide effective delivery of progranulin or at least one granulin protein across the BBB. Additionally, there is also a need for a compound that can provide progranulin with a long enough half-life to persist in in the brain and/or CSF.

While compounds designed to deliver progranulin variants across the BBB are known, such as in US Patent Application No. 2022/0213155, these compounds include: (1) a progranulin variant, not a naturally occurring sequence, which is designed to interact with sortilin and (2) an Fc dimer as the transferrin receptor 1 (TfR1) binding domain. Each will be discussed herein.

### SUMMARY OF THE INVENTION

Provided herein are compounds to deliver naturally occurring progranulin or a fragment of naturally occurring progranulin across the BBB with a long enough half-life to provide a therapeutic benefit to a patient that has a deficiency of progranulin.

In previous attempts to deliver progranulin across the BBB, progranulin has been modified by replacing residues 574-576 to reduce C-terminus clipping of the progranulin protein and to specifically bind to sortilin (SEQ ID NO. 4).

However, it was unexpectedly discovered that clipping (unintentional separation of portions of the progranulin peptide) can be further reduced by instead attaching an albumin binding domain to the C-terminus of wildtype progranulin. Additionally, it was also unexpectedly discovered that modifying progranulin to induce a secondary interaction with sortilin did not lead to an improvement of transport across the BBB, as shown in FIG. 3. As shown in FIG. 4, the use of an albumin binding domain provided half-life extension of the progranulin domain and/or an increase in the amount of the progranulin variant delivered across the BBB.

Additionally, in previous attempts to deliver progranulin across the BBB, an Fc dimer has been used as the TfR1 binding domain. However, it has been unexpectedly found that a TfR1 binding domain including a Fab region with higher binding affinity to human TfR1 receptors led to increased delivery across the BBB, as shown in FIG. 3.

In total, disclosed herein is a compound comprising a progranulin domain, and a transferrin receptor 1 (TfR1) binding domain that can improve delivery of progranulin or a progranulin fragment across the BBB.

Also provided herein is a progranulin domain, a TfR1 binding domain, and an albumin binding domain that can improve delivery of progranulin, or at least one granulin protein across the BBB and with an improved half-life.

Also provided herein is a progranulin domain and a TfR1 binding domain wherein the TfR1 binding domain comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein
the amino acid sequence of HCDR1 is SEQ ID NO. 45,
the amino acid sequence of HCDR2 is SEQ ID NO. 46,
the amino acid sequence of HCDR3 is SEQ ID NO. 47,
the amino acid sequence of LCDR1 is SEQ ID NO. 48,
the amino acid sequence of LCDR2 is SEQ ID NO. 49, and
the amino acid sequence of LCDR3 is SEQ ID NO. 50.

Also provided herein is a compound comprising a progranulin fragment, wherein the amino acid sequence of the progranulin fragment is SEQ ID NO. 2. The progranulin fragment can be from 100 residues to 500 residues in length.

Also provided herein is a compound comprising (a) a progranulin domain, X; (b) a TfR1 binding domain, Y, linked to X with a first linker, L₁; and (c) an albumin binding domain, Z, linked to X or Y with a second linker, L₂.

Also provided herein is a compound comprising (a) a progranulin domain; (b) a TfR1 binding domain linked to the progranulin domain with a first linker; and (c) an albumin binding domain linked to the progranulin domain or the TfR1 binding domain with a second linker.

Also provided herein, is a compound comprising an amino acid sequence having at least 90% or at least 95% sequence identity to the amino acid sequence given by SEQ ID NO. 25, which is the heavy chain of H09 Fab-PGRN-C90.43

Also provided herein is a compound, wherein the compound comprises: (a) a heavy chain having at least 90% or at least 95% sequence identity to the amino acid sequence given by SEQ ID NO. 25; and (b) a light chain having at least 90% or at least 95% sequence identity to the amino acid sequence given by SEQ ID NO. 19.

Also provided herein is a compound, wherein the compound comprises: (a) a heavy chain having the amino acid sequence given by SEQ ID NO. 25; and (b) a light chain having the amino acid sequence given by SEQ ID NO. 19.

Also provided herein is a compound comprising an amino acid sequence having at least 90% or at least 95% sequence identity to the amino acid sequence given by SEQ ID NO. 26, which is the heavy chain H09 Fab-PGRNΔpGF-C90.43.

Also provided herein is a compound, wherein the compound comprises: (a) a heavy chain having at least 90% or at least 95% sequence identity to the amino acid sequence given by SEQ ID NO. 26; and (b) a light chain having at least 90% or at least 95% sequence identity to the amino acid sequence given by SEQ ID NO. 19.

Also provided herein is a compound, wherein the compound comprises: (a) a heavy chain having the amino acid sequence given by SEQ ID NO. 26; and (b) a light chain having the amino acid sequence given by SEQ ID NO. 19.

Also provided herein is a compound comprising comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein the HCDR1 comprises SEQ ID NO. 45, the HCDR2 comprises SEQ ID NO. 46, the HCDR3 comprises SEQ ID NO. 47, the LCDR1 comprises SEQ ID NO. 48, the LCDR2 comprises SEQ ID NO. 49, and the LCDR3 comprises SEQ ID NO. 50.

Also provided herein is a compound comprising: (a) a progranulin domain, wherein the amino acid sequence of the progranulin domain is SEQ ID NO. 1 or SEQ ID NO. 2; (b) a transferrin receptor 1 (TfR1) binding domain linked to the progranulin domain with a first linker, wherein the TfR1 binding region comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein the amino acid sequence of HCDR1 is SEQ ID NO. 45, the amino acid sequence of HCDR2 is SEQ ID NO. 46, the amino acid sequence of HCDR3 is SEQ ID NO. 47, the amino acid sequence of LCDR1 is SEQ ID NO. 48, the amino acid sequence of LCDR2 is SEQ ID NO. 49, and the amino acid sequence of LCDR3 is SEQ ID NO. 50; and (c) an albumin binding domain linked to the progranulin domain or the TfR1 binding domain with a second linker.

Also provided herein is a method of treating a disorder, the method comprising administering any of the disclosed compounds to a patient in need thereof, preferably wherein the disorder is neuronal ceroid lipofuscinosis type-11, frontotemporal dementia, Alzheimer's disease, Parkinson's disease, amyotrophic lateral sclerosis, or a combination thereof.

Also provided herein is a compound for the use in treating neuronal ceroid lipofuscinosis type-11, frontotemporal dementia, Alzheimer's disease, Parkinson's disease, amyotrophic lateral sclerosis, or a combination thereof.

Also provided herein is a pharmaceutical composition comprising any of the disclosed compounds for use in treating neuronal ceroid lipofuscinosis type-11, frontotemporal dementia, Alzheimer's disease, Parkinson's disease, amyotrophic lateral sclerosis, or a combination thereof.

Also provided herein is a use of any of the disclosed compounds in the manufacture of a medicament for the treatment of neuronal ceroid lipofuscinosis type-11, frontotemporal dementia, Alzheimer's disease, Parkinson's disease, amyotrophic lateral sclerosis, or a combination thereof.

Also provided herein a composition comprising any one of the disclosed compounds and a pharmaceutically acceptable carrier.

### BRIEF DESCRIPTION OF THE DRAWINGS

FIG. 1 shows that a construct with a progranulin fragment had a prolonged durability relative to a construct with a full length progranulin peptide.

FIG. 2 shows the impact of progranulin length on tau turnover

FIG. 3 shows the impact of TfR shuttle architectures on PGRN delivery across BBB.

FIG. 4 shows the impact of TfR shuttle affinity on delivery of construct across the BBB.

FIG. 5 shows that a TfR binding domain can be a suitable progranulin shuttle, delivering progranulin into neurons in place of a sortilin binding domain.

FIG. 6 shows CNS exposure of Sandwich Ex. 2 in hTfR KI mice

FIG. 7 shows the impact of BBB-crossing PGRN on lipofuscin pathology, neuronal injury, neuroinflammation and lysosomal function in Grn KO mice.

### DETAILED DESCRIPTION OF THE INVENTION

### Compound

The disclosed compounds deliver progranulin, a fragment of progranulin, and/or at least one granulin protein across the BBB with a long enough half-life to persist in in the brain and/or CSF. The disclosed compounds comprise a progranulin domain, a TfR1 binding domain, and optionally an albumin binding domain. The disclosed compounds can also comprise a progranulin domain, X; a TfR1 binding domain, Y, linked to X with a first linker, L₁; and an albumin domain, Z, linked to X or Y with a second linker, L₂, such as shown in Formula I, II, or III.

Y-L₁-X-L₂-Z Formula I.

In some embodiments, the compounds can be represented by Formula I. The TfR1 binding domain, Y, can be linked to the progranulin domain, X, with a first linker, L₁. The TfR1 binding domain can be attached to the progranulin domain at the N-terminus or the C-terminus of the progranulin domain. The albumin binding domain, Z, can be linked to the progranulin domain with a second linker L₂ at the N-terminus or the C-terminus of the progranulin domain. The TfR1 binding domain and the albumin binding domain can be linked at opposite ends of the progranulin domain. For example, the TfR1 binding domain can be linked at or within 5 amino acids of the N-terminus of the progranulin domain while the albumin binding domain can be linked at or within 5 amino acids of the C-terminus of the progranulin domain, or the TfR1 binding domain can be linked at or within 5 amino acids of the C-terminus of the progranulin domain while the albumin binding domain can be linked at or within 5 amino acids of the N-terminus of the progranulin domain.

Z-L₂-Y-L₁-X Formula II. Compound

In some embodiments, the compounds can be represented by Formula II. The progranulin domain, X, can be linked to the TfR1 binding domain, with a first linker, L₁. The progranulin domain can be attached to the TfR1 binding domain at the N-terminus or the C-terminus of the TfR1 binding domain. The albumin binding domain, Z, can be linked to the TfR1 binding domain with a second linker L₂ at the N-terminus or the C-terminus of the progranulin domain. The progranulin domain and the albumin binding domain can be linked at opposite ends of the TfR1 binding domain. For example, the progranulin domain can be linked at or within 5 amino acids of the N-terminus of the TfR1 binding domain while the albumin binding domain can be linked at or within 5 amino acids of the C-terminus of the TfR1 binding domain, or the progranulin domain can be linked at or within 5 amino acids of the C-terminus of the TfR1 binding domain while the albumin binding domain can be linked at or within 5 amino acids of the N-terminus of the TfR1 binding domain.

In some embodiments, the progranulin domain and the albumin binding domain can be linked at either the same end or opposite ends of the TfR1 binding domain.

In some embodiments, the progranulin domain, X, can be linked to the TfR1 binding domain, with a first linker, L₁. The progranulin domain can be attached to the TfR1 binding domain at the N-terminus or the C-terminus of the TfR1 binding domain. The albumin binding domain, Z, can be linked to the TfR1 binding domain with a second linker L₂ at the N-terminus or the C-terminus of the progranulin domain. The progranulin domain and the albumin binding domain can be linked at the same end of the TfR1 binding domain. For example, the progranulin domain and the albumin binding domain can be linked at or within 5 amino acids of the N-terminus or the C-terminus of the TfR1 binding domain.

In some embodiments, the progranulin domain can be linked to the heavy chain or the light chain of the TfR1 binding domain.

In some embodiments, the compounds are made from one or more polypeptide and/or protein sequences. Thus, in some embodiments, the compound can also be a fusion protein. In some embodiments, the compound is a polypeptide, a protein, and/or a fusion protein.

In some embodiments, any two of the progranulin domain, the TfR1 binding domain, and/or albumin binding domain do not form a dimer. For example, in some embodiments, even when the TfR1 binding domain and the albumin binding domain are at the same end of the progranulin domain, they do not interact to form a TfR1 binding domain-albumin binding domain dimer. The dimer can be a heterodimer, such as an Fc heterodimer.

In some embodiments, the compound comprises a heavy chain comprising SEQ ID NO. 25, SEQ ID NO. 26, and/or SEQ ID NO. 29. In some embodiments, the compound comprises light chain comprising SEQ ID NO. 19, SEQ ID NO. 32, and/or SEQ ID NO. 33.

In some embodiments, the compound comprises a heavy chain comprising SEQ ID NO. 25. In some embodiments, the compound comprises a light chain comprising SEQ ID NO. 19.

In some embodiments, the compound comprises a heavy chain comprising SEQ ID NO. 26. In some embodiments, the compound comprises a light chain comprising SEQ ID NO. 19.

In some embodiments, the compound comprises a heavy chain comprising SEQ ID NO. 29. In some embodiments, the compound comprises a light chain comprising SEQ ID NO. 32.

In some embodiments, the compound comprises a heavy chain comprising SEQ ID NO. 29. In some embodiments, the compound comprises a light chain comprising SEQ ID NO. 33.

In some embodiments, the compound comprises a heavy chain with an amino acid sequence having at least 90%, 95%, and/or 99% sequence identity to the amino acid sequence given by SEQ ID NO. 25, SEQ ID NO. 26, and/or SEQ ID NO. 29.

In some embodiments, the compound comprises a light chain with an amino acid sequence having at least 90%, 95%, and/or 99% sequence identity to the amino acid sequence given by SEQ ID NO. 19, SEQ ID NO. 32, and/or SEQ ID NO. 33.

### Progranulin Domain

The disclosed compounds include a progranulin domain. The progranulin domain is the portion of the disclosed compounds that include the amino acid sequence representing at least one unmodified, naturally occurring, and/or wildtype granulin protein. Progranulin is a precursor protein for granulin proteins, which are cleaved from the full-length progranulin precursor. Progranulin includes a pro-protein (p) followed by 7 granulin protein sequences: G-F—B-A-C-D-E.

The progranulin domain can include at least one, at least two, at least three, at least four, at least 5, at least 6, or all 7 unmodified granulin protein(s).

The progranulin domain can include the full-length unmodified progranulin sequence including the pro-protein and all 7 granulin proteins: p-G-F—B-A-C-D-E, which is described in SEQ. ID NO. 1.

The progranulin domain can include fragments of the full-length sequence of progranulin. For example, the progranulin domain can include the progranulin fragment known as B-A-C-D-E, or PGRNΔpGF, which is described in SEQ. ID NO. 2. The progranulin domain can also include other fragments of progranulin, such as, for example, p-G-F, G-F—B-A-C-D-E, among others.

The progranulin domain can include an unmodified, wild-type, and/or naturally occurring progranulin sequence or a fragment thereof. In some embodiments, the progranulin domain does not include and/or is free of modified granulin protein sequences that do not naturally exist.

It has been unexpectedly found that when the progranulin domain included a fragment of progranulin, i.e. a progranulin fragment or PGRNΔpGF, which is described in SEQ. ID NO. 2, the compound remained stable longer than when the progranulin domain included a full length progranulin sequence. While not wishing to being bound by theory, it is believed that the fragment of full length progranulin, PGRNΔpGF, had a longer stability due to a decrease in clipping present in a smaller progranulin domain.

The progranulin fragment can have a length of less than 550 residues, less than 500 residues, from 100 residues to 500 residues, from 350 residues to 450 residues, from 400 residues to 425 residues, or 414 residues.

### TfR1 Binding Domain

The disclosed compounds can also include a transferrin receptor 1 (TfR1) binding domain. The TfR1 binding domain is a portion of the compound that specifically binds to a TfR1 receptor and/or a human TfR1 receptor. TfR1 receptors are found in neurons and glial cells, thus, while not wishing to being bound by theory, it is believed that attaching a TfR1 binding domain with high binding affinity to TfR1 receptors to a progranulin domain, the penetration across the blood brain barrier (BBB) is enhanced relative to a progranulin sequence that is not attached to a TfR1 binding domain. It is further believed that if delivery of the progranulin domain across the BBB is enhanced, it can allow for peripheral dosing to a patient deficient in progranulin.

The TfR1 binding domain can be a peptide, a protein, an antibody, a fragment of an antibody, a Fc region, a Fab region, a single domain antibody, or combinations thereof. The TfR1 binding domain can include a Fab region. The TfR1 binding domain can also not include and/or be free of an Fc region.

The TfR1 binding domain can also be described by its affinity to a TfR1 receptor. The TfR1 binding domain can have an affinity to a human TfR1 receptor of from about 1 nM to about 100 nM, from about 1 nM to about 50 nM, less than 100 nM, greater than 1 nM to less than 20 nM, from about 5 nM to less than 20 nM, from about 5 nM to about 20 nM, about 10 nM, or 10 nM. Preferably, the affinity of the TfR1 binding domain can be about 10 nM or 10 nM. Normally, it would be expected that the transport across the BBB would be maximized as the affinity concentration of the TfR1 binding domain decreases. Unexpectedly, it has been found that transport across the BBB is maximized when the affinity is less than 20 nM, but greater than 1 nM or about 10 nM.

In some embodiments, the TfR1 binding domain can have a heavy chain comprising SEQ ID NO. 15, SEQ ID NO. 16, SEQ ID NO. 17, and/or SEQ ID NO. 18. In some embodiments, the TfR1 binding domain can have a light chain comprising SEQ ID NO. 19, SEQ ID NO. 20, SEQ ID NO. 21, and/or SEQ ID NO. 22.

In some embodiments, the TfR1 binding domain has a heavy chain comprising SEQ ID NO. 15 and a light chain comprising SEQ ID NO. 19.

In some embodiments, the TfR1 binding domain has a heavy chain comprising SEQ ID NO. 16 and a light chain comprising SEQ ID NO. 20.

In some embodiments, the TfR1 binding domain has a heavy chain comprising SEQ ID NO. 17 and a light chain comprising SEQ ID NO. 21.

In some embodiments, the TfR1 binding domain has a heavy chain comprising SEQ ID NO. 18 and a light chain comprising SEQ ID NO. 22.

In some embodiments, the TfR1 binding domain can include an amino acid sequence having at least 90%, 95%, and/or 99% sequence identity to the amino acid sequence given by SEQ ID NO. 15, SEQ ID NO. 16, SEQ ID NO. 17, SEQ ID NO. 18, SEQ ID NO. 19, SEQ ID NO. 20, SEQ ID NO. 21, and/or SEQ ID NO. 22.

In some embodiments, a suitable TfR1 binding domain can be defined by its complementary determining regions (CDRs), which are the regions of the domain that are believed to interact with the TfR1 receptor. Suitable CDRs for the heavy chain of the TfR1 binding domain can comprise SEQ ID NO. 45, SEQ ID NO. 46, and/or SEQ ID NO. 47. Suitable CDRs for the light chain of the TfR1 binding domain can comprise SEQ ID NO. 48, SEQ ID NO. 49, and/or SEQ ID NO. 50.

In some embodiments, the TfR1 binding domain comprises one or more of SEQ ID NO. 45-50.

In some embodiments, the TfR1 binding domain comprises SEQ ID NO. 45, SEQ ID NO. 46, SEQ ID NO. 47, SEQ ID NO. 48, SEQ ID NO. 49 and SEQ ID NO. 50.

some embodiments, a suitable TfR1 binding domain can comprise the CDRs described in TABLE A.

TABLE A
Example Sequences of CDRs that Bind TfR1

| | | |
|---|---|---|
| HCDR1 | SEQ ID NO. 45 | VASGFTFSSYSMN |
| HCDR2 | SEQ ID NO. 46 | SISSSSSYIYYADSVKG |
| HCDR3 | SEQ ID NO. 47 | ARRHGYSNSDAFDN |
| LCDR1 | SEQ ID NO. 48 | RASQGISHYLV |
| LCDR2 | SEQ ID NO. 49 | YAASSLQS |
| LCDR3 | SEQ ID NO. 50 | LQHNSYPWT |

CDRs of TABLE A are defined as described in North, with the exception of HCDR2 which is defined as described in Kabat. (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)).

TABLE A-1
Example Sequences of CDRs that Bind TfR1

| Ab | North | Kabat | IMGT | Chothia |
|---|---|---|---|---|
| HCDR1 | VASGFTFSSYSMN | SYSMN | GFTFSSYS | GFTFSSY |
| | SEQ ID NO. 45 | SEQ ID NO. 57 | SEQ ID NO. 62 | SEQ ID NO. 68 |
| HCDR2 | SISSSSSYIY | SISSSSSYIYYADSVKG | ISSSSSYI | SSSSSY |
| | SEQ ID NO. 56 | SEQ ID NO. 46 | SEQ ID NO. 63 | SEQ ID NO. 69 |
| HCDR3 | ARRHGYSNSDAFDN | RHGYSNSDAFDN | ARRHGYSNSDAFDN | RHGYSNSDAFDN |
| | SEQ ID NO. 47 | SEQ ID NO. 58 | SEQ ID NO. 64 | SEQ ID NO. 70 |
| LCDR1 | RASQGISHYLV | RASQGISHYLV | QGISHY | RASQGISHYLV |
| | SEQ ID NO. 48 | SEQ ID NO. 59 | SEQ ID NO. 65 | SEQ ID NO. 71 |
| LCDR2 | YAASSLQS | AASSLQS | AAS | AASSLQS |
| | SEQ ID NO. 49 | SEQ ID NO. 60 | SEQ ID NO. 66 | SEQ ID NO. 72 |
| LCDR3 | LQHNSYPWT | LQHNSYPWT | LQHNSYPWT | LQHNSYPWT |
| | SEQ ID NO. 50 | SEQ ID NO. 61 | SEQ ID NO. 67 | SEQ ID NO. 73 |

The CDRs of TABLE A-1 are defined according to methods well known to a person of ordinary skill in the art including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGene Tics database available on at www.imgt.org; see Lefranc et al., Nucleic Acids Res. 1999; 27:209-212).

### Albumin Binding Domain

The disclosed compounds can also include an albumin binding domain. The albumin binding domain is a portion of the compound that specifically binds albumin and/or human serum albumin. In some embodiments, the albumin binding domain binds human serum albumin, but also binds to serum albumins of other species, such as, but not limited to, mouse, rat, and cynomolgus monkey.

It has been expectedly found that by attaching the albumin binding domain to the C-terminus of the progranulin domain, that the half-life of the progranulin domain with be improved and/or the transport of the progranulin domain across the BBB will be improved. While not wishing to being bound by theory, it is believed that the half-life of the progranulin domain is improved because the albumin binding domain attachment at the C-terminus of the progranulin domain will prevent C-terminus clipping of the progranulin domain and/or allow for the progranulin to be recycled through the albumin turnover process.

In some embodiments, the albumin binding domain is attached to the C-terminus of the progranulin domain through a linker, such as L₁ or L₂.

The albumin binding domain can be a peptide, a protein, an antibody, a fragment of an antibody, a Fc region, a Fab region, a single domain antibody, or combinations thereof. Preferably, the albumin binding domain can be a single domain antibody, such as a V_{H}H.

In some embodiments, the albumin binding domain can be represented by SEQ ID NO. 23 or SEQ ID NO. 24.

In some embodiments, the albumin binding domain can include an amino acid sequence having at least 90%, 95%, and/or 99% sequence identity to the amino acid sequence given by SEQ ID NO. 23 or SEQ ID NO. 24.

In some embodiments, a suitable albumin binding domain can be defined by its complementary determining regions (CDRs), which are the regions of the domain that are believed to interact with albumin. Suitable CDRs for the albumin binding domain can comprise SEQ ID NO. 51, SEQ ID NO. 52, and/or SEQ ID NO. 53.

In some embodiments, the TfR1 binding domain comprises SEQ ID NO. 51, SEQ ID NO. 52, and SEQ ID NO. 53.

In some embodiments, a suitable albumin binding domain can comprise the CDRs described in TABLE B.

TABLE B
Example Sequences of CDRs that Bind Albumin

| | | |
|---|---|---|
| CDR1 | SEQ ID NO. 51 | AASGRYIDETAVA |
| CDR2 | SEQ ID NO. 52 | GIGGGVDITYYADSVKG |
| CDR3 | SEQ ID NO. 53 | GARPGRPLITSKVADLYPY |

CDRs are defined as described in North, with the exception of CDR2 which is defined as described in Kabat. (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)).

### Linker

The disclosed compounds include one or more linkers, L₁, L₂, L₃, etc. The linkers are used to connect the progranulin domain, the TfR1 binding domain, and the albumin binding domain to one another. In some embodiments, the disclosed compounds have one linker, two linkers, three linkers, or from one to three linkers.

In some embodiments, a first linker, L₁, connects the TfR1 binding domain, Y, to the progranulin domain, X. In some embodiments, a second linker, L₂, connects the albumin binding domain, Z, to the progranulin domain, X, or the TfR1 binding domain, Y. In some embodiments, L₁ and L₂ are identical. In some embodiments, L₁ and L₂ are not identical.

The linker(s) can independently comprise a covalent bond, a peptide linker, a PEG linker, a disulfide bond, a thioacetal linkage, or a thioester linkage. The linker(s) can be independently selected from covalent bond, a peptide linker, a PEG linker, a disulfide bond, a thioacetal linkage, and a thioester linkage.

In some embodiments, the linker(s) is a peptide linker. Suitable peptide linkers include peptides of from 2 to 50 amino acids in length. Other suitable peptide linkers include (G4U)ₙ, wherein U is any suitable amino acid and n is a whole digit integer from 1 to 10. Suitable amino acids that can be represented by U include glutamine (Q), serine(S), asparagine (N), alanine (A), among others. Preferably, the linker(s) is (G4Q)ₙ or (G4S)ₙ wherein n is from 3 to 5.

In some embodiments, the linker(s) can be represented by one or more of SEQ ID NO. 5-14.

In some embodiments the first linker and the second linker can be represented by SEQ ID NO. 7.

In some embodiments, the linker(s) is a PEG linker. Suitable PEG linkers include those represented by Formula III, wherein n is a whole number integer from 1 to 10.

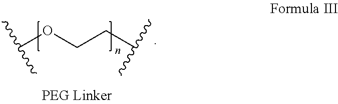

Other suitable peptide linkers include DKT(G₄U)ₙ, wherein U is any suitable amino acid and n is a whole digit integer from 1 to 10. Suitable amino acids that can be represented by U include glutamine (Q), serine(S), asparagine (N), alanine (A), among others. In some embodiments, the linker(s) is DKT(G₄Q)ₙ or DKT(G₄S), wherein n is from 3 to 5.

### Compositions and Routes of Administration

The compounds of the present invention can be used as medicaments in human medicine, administered by a variety of routes. Most preferably, such compositions are for parenteral administration. Such pharmaceutical compositions can be prepared by methods well known in the art (See, e.g., Remington: The Science and Practice of Pharmacy, 19th ed. (1995), A. Gennaro et al., Mack Publishing Co.) and comprise the compounds as disclosed herein, and a pharmaceutically acceptable carrier, diluent, or excipient.

### Methods of Treatment

The compound of the present disclosure can be used in aiding in the treatment of patients, particularly for aiding in treatment of CNS diseases, including neurodegenerative diseases, such as neuronal ceroid lipofuscinosis type-11, frontotemporal dementia, Alzheimer's disease, Parkinson's disease, amyotrophic lateral sclerosis (ALS), among others. Treatment includes administration of a compound of the present disclosure for the treatment of a CNS disease or condition in a human that would benefit from delivering the progranulin domain across the blood brain barrier to mitigate the impact of lower levels of progranulin in a patient.

### Definitions

The term “antibody,” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An exemplary antibody of the present disclosure is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)2, Fv fragments, scFv antibody fragments, scFab, disulfide-linked Fvs (sdFv), a Fd fragment.

The terms “bind” and “binds” as used herein are intended to mean, unless indicated otherwise, the ability of a binding domain of a compound to form a chemical bond or attractive interaction with another protein or molecule, which results in proximity of the two proteins or molecules as determined by common methods known in the art.

The term “progranulin domain” refers to a portion of a compound, or polypeptide sequence, of the present disclosure that includes the amino acid sequence representing at least one unmodified, or wildtype granulin protein. In some embodiments, the progranulin domain can include the amino acid sequence representing up to all seven granulin proteins and the pro-protein naturally contained in progranulin. In some embodiments, the progranulin domain can include the amino acid sequence representing more than one, but less than the seven granulin proteins and the pro-protein naturally contained in progranulin, i.e. a progranulin fragment. In one non-limiting example, the progranulin domain is represented by the amino acid sequence corresponding to SEQ ID NO. 1, SEQ ID NO. 2 and/or SEQ ID NO. 3. In another non-limited example, the progranulin domain excludes SEQ ID NO. 4.

The term “TfR1 binding domain” refers to an antibody, a portion of an antibody, a portion of a compound, or a polypeptide sequence, that binds a transferrin receptor, i.e. TfR1.

The term “albumin binding domain” refers to an antibody, a portion of an antibody, a portion of a compound, or a polypeptide sequence, that binds albumin. In one non-limiting example, albumin binding domain refers to a portion of a compound of the present disclosure that binds to human serum albumin.

As used interchangeably herein, the term “patient,” “subject,” and “individual,” refers to a human. In certain embodiments, the patient is further characterized with a CNS disease, disorder, or condition (for example, a CNS neurodegenerative disorder). In some embodiments, the patient may be further characterized as being at risk of developing a CNS disorder, disease, or condition.

As used herein, a “Fab” means a fragment antigen-binding region of an antibody. Additionally, as used herein, the Fab region includes a heavy chain, which is linked to another portion of the disclosed compound, such as a linker, progranulin domain, TfR1 binding domain, and/or albumin binding domain. The Fab region also includes a light chain, which is covalently bonded to the heavy chain.

As used herein, a “single-domain antibody” is an antibody fragment consisting of a single monomeric variable antibody chain. The single-domain antibody includes an antigen-binding region.

As used herein, a “V_{H}H fragment” is a single-domain antibody that is engineered from heavy-chain antibodies found in camelids.

As used herein, the term “peptide” or “peptide chain”, refers to a polymer comprising two (2) or more amino acids and/or amino acid derivatives which, in general, are linked via peptide bonds. Embodiments of peptides may include modifications or amino acid derivatives, including post-translational modifications such as, phosphorylation, hydroxylation, sulfonation, palmitoylation, glycosylation and disulfide formation.

The term, “linked to” or “linked with”, as used herein, refers to a first nucleotide (or polynucleotide) or peptide being associated, attached, connected or otherwise joined to a second nucleotide (or polynucleotide) or peptide. For example, a first polynucleotide can be linked to a second polynucleotide sequence such that they form a fusion peptide or protein when the sequence is translated. Likewise, a first peptide sequence can be linked to a second peptide sequence via covalent or non-covalent interactions to form a multimeric peptide. Alternatively, “linked to” or “linked with” refers to a nucleotide (or polynucleotide sequence) or peptide that is associated, connected or joined to a non-nucleotide or non-peptide moiety. For example, a peptide may be linked to a fatty acid moiety (i.e., acylated) to form a conjugated peptide.

The term “conjugate group,” as used herein, refers to a group that is attached or linked to a peptide. Conjugate groups can include a conjugate moiety and a conjugate linker for attaching or linking the conjugate moiety to the peptide.

The term “conjugate linker,” as used herein, refers to an atom, group of atoms, molecule or compound (such as an amino acid or group of amino acids) comprising at least one bond that attaches or links a conjugate moiety to a peptide herein.

The term “conjugate moiety,” as used herein, means a molecule or compound, especially a non-peptide molecule or compound, that is attached or linked to a peptide herein either directly or via a conjugate linker.

### EXAMPLES

### Example 1—Expression of Disclosed Compounds

Compounds of the present invention can be expressed essentially as follows. An appropriate host cell, such as HEK 293 or CHO, can be either transiently or stably transfected with an expression system for secreting antibodies using an optimal predetermined HC:LC vector ratio (such as 1:3, 1:2, 1:1) or a single vector system encoding both the HC and the LC.

### Purification of Kappa Designs

To assess the purification properties of the CI pool, IL of sCHO supernatant containing the CI were purified using a 3-column process. Supernatant was applied to a ˜60 mL prepacked CaptoL resin column preequilibrated with 20 mM Tris (pH 7.0). Column with loaded supernatant was subsequently washed with 5 column volumes of 20 mM Tris (pH 7.0). mAb was eluted from CaptoL resin by applying 5 column volumes of a mixed acid elution buffer (20 mM Acetic acid, 5 mM Citric acid). CaptoL elution pool was neutralized to pH 5 with 0.5 M Tris base, centrifuged and applied to a 0.22-micron sterile filter. This CaptoL purified material was further purified by PrismA resin affinity purification. CaptoL pool was adjusted to pH˜7 using 1 M Tris pH7.5 and was applied to a 58 mL PrismA prepacked column preequilibrated in 50 mM Tris (pH8.0), washed with 5 column volumes of equilibration buffer, and eluted with 5 column volumes of a mixed acid elution buffer (20 mM Acetic acid, 5 mM Citric acid). PrismA pool was neutralized to pH˜5 using 0.5 M Tris base. Pool was further purified using Cation Exchange chromatography (CEX). CaptoL pool was loaded onto a 70 mL prepacked Poros HS50 CEX column equilibrated in 20 mM Acetate, pH5, and eluted with same buffer supplemented with 1 M NaCl with a 5-60% linear gradient over 15 column volumes. Subsequently, eluted fractions containing monomer were pooled, sterile filtered and dialyzed into PBS overnight at 4° C. Following dialysis, protein was sterile filtered and protein concentration/yield was determined at A280 using the calculated extinction coefficient. This sCHO material was used to all developability and HCP protein analysis.

TABLE 1
Kappa Designs Expressed and Purified

| | | | TfR1 | Albumin |
|---|---|---|---|---|
| | | | Binding | Binding |
| Construct | Description | Linker | Domain | Domain |
| Kappa | Heavy Chain: H09-C90.43 (SEQ ID NO. 29) | (G4Q)₅ | H09 | C90.43 |
| Ex. 1 | Light Chain: H09 PGRNdesQLL | | | |
| | (SEQ ID NO. 30) | | | |
| Kappa | Heavy Chain: H09-C90.43 (SEQ ID NO. | (G4Q)₅ | H09 | C90.43 |
| Ex. 2 | 29) | | | |
| | Light Chain: H09 PGRNΔpGFdesQLL | | | |
| | (SEQ ID NO. 31) | | | |
| Kappa | Heavy Chain: H09-C90.43 (SEQ ID NO. 29) | (G4Q)₅ | H09 | C90.43 |
| Ex. 3 | Light Chain: H09 PGRN (SEQ ID NO. 32) | | | |
| Kappa | Heavy Chain: H09-C90.43 (SEQ ID NO. | (G4Q)₅ | H09 | C90.43 |
| Ex. 4 | 29) | | | |
| | Light Chain: H09 PGRNΔPGF | | | |
| | (SEQ ID NO. 33) | | | |
| Kappa | Heavy Chain: 10E10-C80.43 (SEQ ID NO. | (G4Q)₅ | 10E10 | C80.43 |
| Ex. 5 | 34) | | | |
| | Light Chain: 10E10-PGRN 10 nM TfR | | | |
| | affinity (SEQ ID NO. 35) | | | |
| Kappa | Heavy Chain: 10E10-C80.43 (SEQ ID NO. | (G4Q)₅ | 10E10 | C80.43 |
| Ex. 6 | 34) | | | |
| | Light Chain: 10E10-PGRNΔpGF | | | |
| | (SEQ ID NO. 36) | | | |
| Kappa | Heavy Chain: 10E10-C80.43 (SEQ ID NO. | (G4Q)₅ | 10E10 | C80.43 |
| Ex. 7 | 34) | | | |
| | Light Chain: 10E10-PGRN 300 nM TfR | | | |
| | affinity (SEQ ID NO. 37) | | | |
| Kappa | Heavy Chain: 10E10-C80.43 (SEQ ID NO. | (G4Q)₅ | 10E10 | C80.43 |
| Ex. 8 | 34) | | | |
| | Light Chain: 10E10-PGRN 1000 nM TfR | | | |
| | affinity (SEQ ID NO. 38) | | | |

### Purification of Sandwich Designs

To assess the purification properties of the CI pool, 3 L of sCHO supernatant containing the CI were purified using a 3-column process. Supernatant was applied to a ˜138 mL prepacked PrismA resin column preequilibrated with 50 mM Tris (pH 8.0). Column with loaded supernatant was subsequently washed with 5 column volumes of 50 mM Tris (pH 8.0). mAb was eluted from PrismA resin by applying 5 column volumes of a mixed acid elution buffer (20 mM Acetic acid, 5 mM Citric acid). PrismA elution pool was neutralized to pH 5 with 0.5 M Tris base, centrifuged and applied to a 0.22-micron sterile filter. This PrismA purified material was further purified by CaptoL resin affinity purification. PrismA pool was adjusted to pH˜7 using 1 M Tris pH7.5 and was applied to a 120 mL CaptoL prepacked column preequilibrated in 20 mM Tris pH7, washed with 5 column volumes of equilibration buffer, and eluted with 5 column volumes of a mixed acid elution buffer (20 mM Acetic acid, 5 mM Citric acid). CaptoL pool was neutralized to pH˜5 using 0.5 M Tris base. Pool was further purified using Cation Exchange chromatography (CEX). CaptoL pool was loaded onto a 70 mL prepacked Poros HS50 CEX column equilibrated in 20 mM Acetate, pH5, and eluted with same buffer supplemented with 1 M NaCl with a 5-60% linear gradient over 15 column volumes. Subsequently, eluted fractions containing monomer were pooled, sterile filtered and dialyzed into PBS overnight at 4° C. Following dialysis, protein was sterile filtered and protein concentration/yield was determined at A280 using the calculated extinction coefficient. This sCHO material was used to all developability and HCP protein analysis.

TABLE 2
Sandwich Designs Expressed and Purified

| | | | TfR1 | Albumin |
|---|---|---|---|---|
| | | | Binding | Binding |
| Construct | Description | Linker¹ | Domain | Domain |
| Sandwich | Heavy Chain: H09-PGRN- | (G4Q)₃ | H09 | C90.43 |
| Ex. 1 | C90.43 (SEQ ID NO. 25) | | | |
| | Light Chain: H09 Fab | | | |
| | (SEQ ID NO. 19) | | | |
| Sandwich | Heavy Chain: H09- | (G4Q)₃ | H09 | C90.43 |
| Ex. 2 | PGRNΔpGF-C90.43 | | | |
| | (SEQ ID NO. 26) | | | |
| | Light Chain: H09 Fab | | | |
| | (SEQ ID NO. 19) | | | |
| Sandwich | Heavy Chain: 8D3-PGRN- | (G4Q)₃ | 8D3 | C90.43 |
| Ex. 3 | C90.43 (SEQ ID NO. 27) | | | |
| | Light Chain: 8D3 Fab | | | |
| | (SEQ ID NO. 20) | | | |
| Sandwich | Heavy Chain: H3.03-PGRN- | (G4Q)₃ | H3.03 | C90.43 |
| Ex. 4 | C90.43 (SEQ ID NO. 28) | | | |
| | Light Chain: H3.03 Fab | | | |
| | (SEQ ID NO. 21) | | | |

¹Linker used for Connection Between TfR1 Binding Domain//Progranulin Domain and Progranulin Domain//Albumin Binding Domain

### Expression and Purification of Comparative Example 1

Comparative Ex. 1 was expressed and purified as described in WO2019246071A1 (SEQ ID 227 and 291 in WO2019246071A1).

TABLE 3
Fc Constructs

| | | | TfR1 | Albumin |
|---|---|---|---|---|
| | | | Binding | Binding |
| Construct | Description | Linker | Domain | Domain |
| Comparative | SEQ ID NO. 41 and | (G4S)₂ | Fc | N/A |
| Ex. 1 | SEQ ID NO. 42 | | | |
| Comparative | SEQ ID NO. 39 and | (G4S)₅ | Fc | N/A |
| Ex. 2 | SEQ ID NO. 40 | | | |

### Example 2—Transferrin Receptor 1 (TfR1) Binding of H09 Binding Domain

To determine the binding kinetics and affinity of H09-PGRN and BACDE to human TfR1 (hTfR1), human TfR2 (hTfR2) and cynomolgus TfR1, binding affinities and kinetics for antibody to antigen were determined using a BIACORE® T200 instrument. The binding kinetics and affinity of antibodies H09-PGRN and BACDE to human TfR1 ECD were determined using surface plasmon resonance biosensor such as BIAcore® T200 (GE Healthcare, Piscataway, N.J.). Briefly described, BIAcore® T200 instrument is used to measure the binding kinetics for Sandwich Ex. 1 (H09-PGRN-C90.43) and Sandwich Ex. 2 (H09-PGRNΔpGF-C90.43 to hTfR1, hTfR2 and cyno Tfr1 via surface plasmon resonance (SPR) at 25° C. Samples are dissolved in 1×HBS-EP+running buffer (Teknova cat. #H802) and His tagged ECD of the hTfR1, hTfR2, and cyno TfR was amine coupled to the Cytiva CM3 Series S sensor chip.

Human TfR1 ECD protein was generated with a hexa-histidine tag and was expressed in 293 cells and purified by nickel charged IMAC followed by size exclusion chromatography. hTfR2 and cynomolgus TfR ECD were purchased from SYNGENE.

Binding is evaluated using multiple analytical cycles. Each cycle is performed at 25° C. at a flow rate of 50 μl/min for ligand association and dissociation. Each kinetic cycle consists of the following steps: injection of the Sandwich Ex. 1 (H09-PGRN-C90.43) and Sandwich Ex. 2 (H09-PGRNΔpGF-C90.43) over all four flow cells (starting at 125 nM and using two-fold serial dilutions for each cycle) for 240 seconds and followed by 900 seconds for dissociation phase, and regeneration using 3 M MgCl2 hydrochloride over a 30 s contact time. Association (i e., kₒₙ) and dissociation rates (i.e., K_{off}) for each are evaluated using standard double referencing and fit to “1:1 (Langmuir) binding” model in the BiaEvaluation software in batch mode. The affinity (KD) is calculated from the binding kinetics according to the relationship K)=Koff/Kon.

In experiments performed as described protein constructs of the invention exhibited following affinities to TfR1 ligands displayed in TABLE 4. As shown in TABLE 4, both Sandwich Example 1 and Sandwich Ex. 2 displayed 1:1 binding.

TABLE 4
TfR1 Affinity

| | ka | | | | |
|---|---|---|---|---|---|
| Sample | (1/Ms) | kd (1/s) | KD (M) | Ligand | Model |
| Sandwich | 595000 | 0.00434 | 7.29E−09 | hTfR1 | 1:1 Binding |
| Ex. 1 | | | | | |
| Sandwich | 485000 | 0.0047 | 9.7E−09 | cyno TfR1 | 1:1 Binding |
| Ex. 1 | | | | | |
| Sandwich | 730000 | 0.0052 | 7.12E−09 | hTfR1 | 1:1 Binding |
| Ex. 2 | | | | | |
| Sandwich | 244000 | 0.00318 | 1.3E−08 | cyno TfR1 | 1:1 Binding |
| Ex. 2 | | | | | |

### Example 3—PD Durability in Grn KO Mice

Bis(monoacylglycero) phosphate (BMP) is a structural lipid important for lysosomal protein membrane docking and function has been proposed as key biomarker of lysosomal dysfunction. PGRN is critically involved in maintaining BMP levels in the lysosomes (Logan et al, Cell, 2021, Boland et al, Nat Comm, 2022). To evaluate the impact of PGRN variants on the rescue of BMP deficiency, 3-4 months old Grn KO mice were treated with 10E10 Kappa PGRN (Kappa Ex. 5, HC: SEQ ID NO. 34 and LC: SEQ ID NO. 35) or 10E10 Kappa PGRNΔpGF (Kappa Ex. 6, HC: SEQ ID NO. 34 and LC: SEQ ID NO. 36) intravenously (i.v.) at 1.5, 5 and 15 mg per kg. Wild-type littermates and Grn KO mice without treatment were used as positive and negative control respectively. Brain BMP 22:6 levels were assessed at day 1, 4, 7, 14, 21, 35 and 63 post treatment.

TABLE 5
Brain 22:6 BMP levels relative to internal standard after Kappa PGRN or Kappa PGRNΔpGF
treatment in Grn KO mice. Values represent mean +/− standard deviation.

| Day post | Kappa Ex. 5 | | | Kappa Ex. 6 | | | | Wild type |
|---|---|---|---|---|---|---|---|---|
| treatment | 1.5 mpk | 5 mpk | 15 mpk | 1.5 mpk | 5 mpk | 15 mpk | Untreated | control |
| 1 | 0.70 ± 0.07 | 1.01 ± 0.09 | 0.95 ± 0.08 | 0.90 ± 0.05 | 0.87 ± 0.01 | 0.93 ± 0.03 | 1.1 ± 0.13 | 0.6 ± 0.12 |
| 4 | 1.00 ± 0.14 | 1.10 ± 0.04 | 1.15 ± 0.09 | 1.01 ± 0.05 | 1.22 ± 0.22 | 1.19 ± 0.05 | | |
| 7 | 0.89 ± 0.09 | 1.06 ± 0.10 | 1.15 ± 0.06 | 1.02 ± 0.07 | 1.10 ± 0.05 | 1.14 ± 0.10 | | |
| 14 | 0.87 ± 0.05 | 0.98 ± 0.03 | 1.26 ± 0.17 | 0.95 ± 0.09 | 1.13 ± 0.08 | 1.16 ± 0.17 | | |
| 21 | 0.83 ± 0.02 | 1.04 ± 0.10 | 1.18 ± 0.17 | 1.04 ± 0.06 | 1.20 ± 0.16 | 1.26 ± 0.02 | | |
| 35 | 0.73 ± 0.02 | 0.90 ± 0.06 | 0.92 ± 0.07 | 0.88 ± 0.04 | 1.03 ± 0.09 | 1.21 ± 0.03 | | |
| 63 | 0.60 ± 0.03 | 0.67 ± 0.02 | 0.77 ± 0.03 | 0.75 ± 0.06 | 0.74 ± 0.004 | 0.82 ± 0.01 | | |

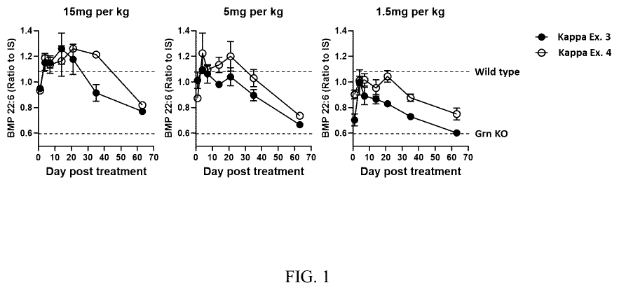

TABLE 5 and FIG. 1. shows that the construct with a progranulin fragment (PGRNΔpGF, i.e. the progranulin fragment does not include the p pro-protein, G granulin, and F granulin) has a prolonged PD effect and a prolonged durability in inducing a pharmacological response in Grn KO mice relative to a construct with a full length progranulin peptide. While not wishing to being bound by theory, it is believed that a construct including PGRNΔpGF will have a prolonged durability relative to a construct including PGRN.

### Example 4—Tau Turnover

To investigate the impact of BBB-crossing PGRN on tau turnover, wild-type mice were treated with Kappa H09 PGRN (Kappa Ex. 3) and Kappa H09 PGRNΔpGF (Kappa Ex. 4) at 5 mg per kg intravenously. Levels of soluble tau protein were examined 7 days post treatment using ELISA. Statistical significance was determined using one-way ANOVA with correction for multiple comparison.

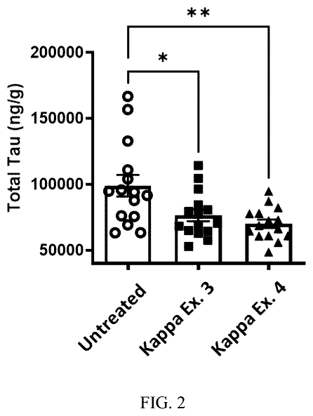

TABLE 7 and FIG. 2 show that both constructs (Full length PGRN and PGRNΔpGF) decreased the tau turnover. Tau turnover is a biomarker that shows that both constructs are delivering progranulin across the BBB in wild type mice.

TABLE 7
Impact of Progranulin Length on Tau turnover

| | Untreated | Kappa Ex. 3 | Kappa Ex. 4 |
|---|---|---|---|
| Total tau | 98917 ± 30511 | 76553 ± 16793 | 70155 ± 11956 |
| (ng/g) | | | |

### Example 5—TfR Shuttle Delivers PGRN Across BBB

To investigate if BBB-crossing PGRN can rescue lipofuscin accumulation in Grn KO mice, 8 months old Grn KO mice were treated with Kapp PGRN or Kappa PGRNΔpGF at various doses and dosing intervals. Brain tissues and CSF were collected 12 weeks post initial dose. To avoid any immunogenicity to testing articles, CD4 T cells were depleted using GK1.5 anti-CD4 monoclonal antibody. Impact of BBB-crossing PGRN on lipofuscine accumulation, activation of microglia and astrocytes and CSF neurofilament light chain (NfL) levels were assessed at 1, 6, 10, 24, 48, 72, and 168 hours post treatment. To estimate PGRN exposure in interstitial fluid and intracellular compartment of the brain, phosphate-buffered saline (PBS) buffer soluble proteins and RIPA buffer (a buffer used to lyse cells and tissues to facilitate isolation of cytoplasmic, membrane, nuclear, and mitochondrial proteins) soluble proteins were serially extracted. PGRN levels in PBS, RIPA fraction of the brain tissues, and cerebrospinal fluid (CSF) were measured using enzyme-linked immunoassay (ELISA). PTV11 (Comparative Ex. 1) was also evaluated as a reference compound.

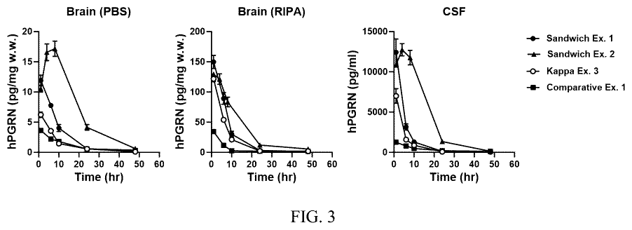

TABLE 8 shows the levels of progranulin delivered across the BBB to the brain and/or CSF after up to 168 hours post treatment with Sandwich Ex. 1. TABLE 9 shows the levels of progranulin delivered across the BBB to the brain and/or CSF after up to 168 hours post treatment with Kappa Ex. 3. Sandwich Ex. 1 and Kappa Ex. 3 have the same components: H09 Fab and C90.43 V_{H}H linked to full length progranulin with (G4Q) 3 peptide linkers. However, in Sandwich Ex. 1, the heavy chain of the H09 Fab antibody portion is connected to the N-terminus of the progranulin sequence and the C90.43 V_{H}H is linked to the C-terminus of the progranulin sequence, as shown in SEQ ID NO. 25. In Kappa Ex. 3, the heavy chain of H09 Fab antibody portion is connected to C90.43 V V_{H}H and the light chain of the H09 Fab antibody portion is connected to the N-terminus of the progranulin sequence. As shown in TABLE 8 and FIG. 3, administration with the Sandwich Ex. 1 led to increased PGRN levels in the brain and CSF relative to the Kappa Ex. 3. This result was consistent throughout the entire post treatment period, up to 168 hours post treatment.

TABLE 10 shows the levels of progranulin delivered across the BBB to the brain and/or CSF after up to 168 hours post treatment with Comparative Ex. 1. Comparative Ex. 1 is a literature compound that was disclosed in U.S. Patent Application Publication No. US 2021/0284702. As shown in TABLE 10 and FIG. 3, administration of both inventive constructs: Sandwich Ex. 1 and Kappa Ex. 3, led to higher levels of progranulin in the brain and CSF relative to administration with Comparative Ex. 1. This result was consistent throughout the entire post treatment period, up to 168 hours post treatment. As such, it is believed that the constructs disclosed herein would have improved delivery of PGRN across the BBB and into the brain and CSF.

TABLE 8A
PGRN exposure in brain and CSF of human TfR KI mice
after treatment with Sandwich Ex. 1. PGRN levels are
expressed as mean ± standard pg/mg brain wet
weight (w.w.) and pg/mL in brain and CSF respectively.

| | H09-PGRN-C90.43 | | |
|---|---|---|---|
| Time post | Sandwich Ex. 1 | | |
| treatment | Brain-PBS | Brain-RIPA | CSF |
| (hr) | (pg/mg w.w.) | (pg/mg w.w.) | (pg/mL) |
| 1 | 11.91 ± 1.54 | 149.82 ± 15.49 | 12420.2 ± 2877.18 |
| 6 | 7.78 ± 0.50 | 89.22 ± 16.85 | 3152.94 ± 544.97 |
| 10 | 4.03 ± 1.03 | 30.32 ± 8.06 | 1290.79 ± 503.71 |
| 24 | 0.59 ± 0.16 | 3.09 ± 1.11 | 134.69 ± 28.90 |
| 48 | 0.35 ± 0.05 | 1.30 ± 0.40 | 18.44 ± 22.41 |

TABLE 8B
PGRN exposure in brain and CSF of human TfR KI mice
after treatment with Sandwich Ex. 2. PGRN levels are
expressed as mean ± standard pg/mg brain wet
weight (w.w.) and pg/mL in brain and CSF respectively.

| | H09-dPGF-C90.43 | | |
|---|---|---|---|
| Time post | Sandwich Ex. 2 | | |
| treatment | Brain-PBS | Brain-RIPA | CSF |
| (hr) | (pg/mg w.w.) | (pg/mg w.w.) | (pg/mL) |
| 1 | 10.52 ± 0.79 | 129.56 ± 3.86 | 10847.96 ± 246.16 |
| 4 | 16.58 ± 1.96 | 121.39 ± 12.34 | 12739.72 ± 1089.29 |
| 9 | 17.16 ± 1.79 | 83.38 ± 11.33 | 11763.27 ± 1287.38 |
| 24 | 4.14 ± 0.66 | 12.04 ± 1.31 | 1346.26 ± 152.16 |
| 48 | 0.63 ± 0.21 | 5.64 ± 0.86 | 147.01 ± 11.05 |

TABLE 9
PGRN exposure in brain and CSF of human TfR KI mice
after treatment with Kappa Ex. 3. PGRN levels are
expressed as mean ± standard pg/mg brain wet
weight (w.w.) and pg/mlLin brain and CSF respectively.

| | H09 Kappa PGRN-C90.43 | | |
|---|---|---|---|
| | Kappa Ex. 3 | | |
| Time post | Brain-PBS | Brain-RIPA | CSF |
| treatment (hr) | (pg/mg w.w.) | (pg/mg w.w.) | (pg/mL) |
| 1 | 6.22 ± 0.89 | 121.71 ± 81.9 | 7002.73 ± 1277.94 |
| 6 | 3.56 ± 0.08 | 53.90 ± 3.75 | 1579.28 ± 122.82 |
| 10 | 1.47 ± 0.60 | 21.18 ± 6.31 | 880.79 ± 242.21 |
| 24 | 0.57 ± 0.22 | 2.23 ± 1.10 | 76.30 ± 53.40 |
| 48 | 0.11 ± 0.03 | 1.87 ± 0.85 | below LLOQ |

TABLE 10
PGRN exposure in brain and CSF of human TfR KI mice
after treatment with Sandwich Ex. 1. PGRN levels are
expressed as mean ± standard pg/mg brain wet
weight (w.w.) and pg/mL in brain and CSF respectively.

| Time | PTV11 | | |
|---|---|---|---|
| post | Comparative Ex. 1 | | |
| treatment | Brain-PBS | Brain-RIPA | CSF |
| (hr) | (pg/mg w.w.) | (pg/mg w.w.) | (pg/mL) |
| 1 | 3.63 ± 0.15 | 34.40 ± 2.06 | 1257.05 ± 247.95 |
| 6 | 2.21 ± 0.41 | 11.59 ± 1.26 | 793.28 ± 103.39 |
| 10 | 1.82 ± 0.35 | 2.50 ± 0.90 | 480.76 ± 141.24 |
| 24 | 1.57 ± 0.16 | 1.99 ± 0.64 | 205.95 ± 79.08 |
| 48 | 0.31 ± 0.06 | 1.62 ± 1.04 | 124.33 ± 72.58 |

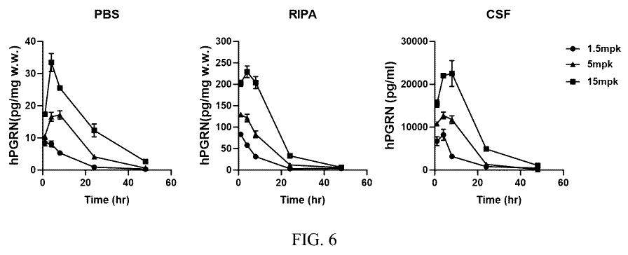

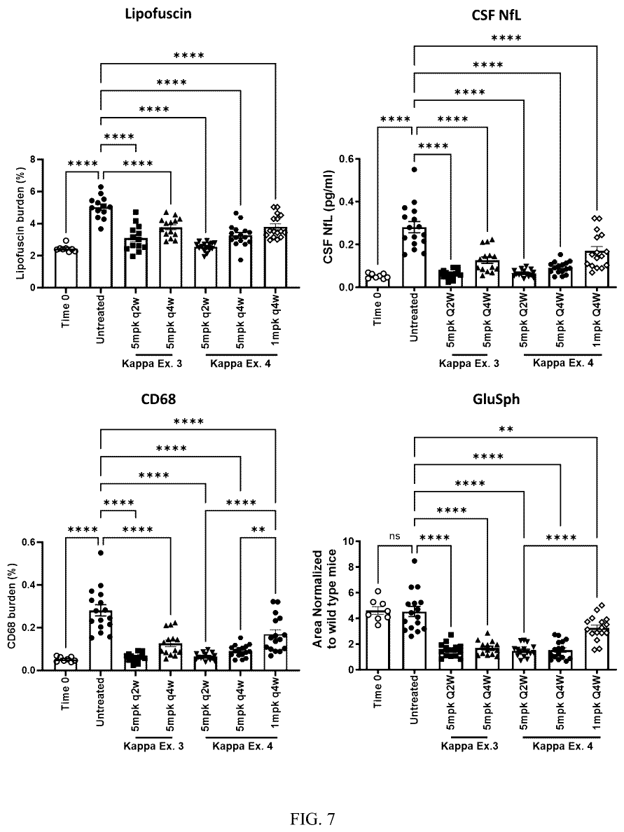

FIG. 6 shows the CNS exposure of Sandwich Ex. 2 in hTfR KI mice and FIG. 7 shows the impact of BBB-crossing PGRN on lipofuscin pathology, neuronal injury, neuroinflammation and lysosomal function in Grn KO mice.

### Example 6—Evaluate TfR1 Shuttle Affinity on PGRN CNS Delivery

To better understand affinity requirement of TfR1 shuttle in delivering PGRN across BBB, Grn−/− mice were treated with three 10E10-PGRN variants with TfR binding at 10 nM (Kappa Ex. 6), 300 nM (Kappa Ex. 7), and 1000 nM (Kappa Ex. 8) at 5 mg per kg. Brain BMP 22:6 levels were assessed at 7 days post treatment. Wild-type littermates and Grn−/− mice without treatment were used as positive and negative control respectively. His tagged PGRN (SYNGENE, SEQ ID NO. 55) was also evaluated as control.

TABLE 11 shows that a wild type mouse had a Brain BMP 22:6 level of 0.74±0.07 and a Grn−/− mouse had a Brain BMP 22:6 level of 0.39±0.02. A peripheral dose of His tagged PGRN (His-PGRN) did not improve the Brain BMP 22:6 level in Grn−/− mice. A construct (Kappa Ex. 8) including a TfR binding domain with a TfR1 affinity of 1000 nM led to a Brain BMP 22:6 level of 0.65=0.09, which indicated that this construct improved the levels of PGRN in the brain better than PGRN alone or without treatment, but the improvement did not approach the amount of PGRN found in Wild Type mice. Unexpectedly, a construct (Kappa Ex. 7) with a TfR1 binding domain with a stronger affinity (300 nM) did not improve the amount of PGRN found in Grn−/− mice. Instead, the construct (Kappa Ex. 7) with the 300 nM shuttle led equivalent levels of PGRN in Grn−/− mice than the levels of PGRN found in Grn−/− mice that were untreated or treated with PGRN without a TfR1 binding domain.

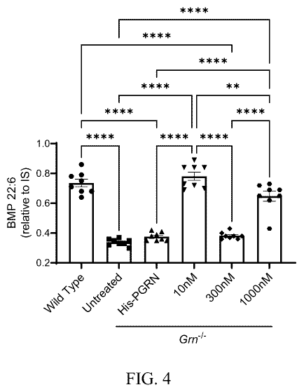

Unexpectedly, a construct (Kappa Ex. 6) with a TfR1 binding domain with a 10 nM affinity to TfR1 did improve the amount of PGRN found in Grn−/− mice. In fact, the levels of PGRN found in Grn−/− mice treated with the construct including a 10 nM TfR1 binding domain were similar to the positive control (Wild Type mice). The data in TABLE 11 and FIG. 4 demonstrate that a construct with a TfR1 binding domain having an affinity of about 10 nM will lead to transport of the attached progranulin domain across the BBB to improve the deficiency of PGRN levels seen in Grn−/− mice.

TABLE 11
Brain 22:6 BMP levels relative to internal standard. Values
represent mean +/− standard deviation.

| | Grn^{-/-} | | | | |
|---|---|---|---|---|---|
| | | | Kappa | Kappa | Kappa |
| Wild | | His- | Ex. 6 | Ex. 7 | Ex. 8 |
| Type | Untreated | PGRN | 10 nM | 300 nM | 1000 nM |
| 0.74 ± | 0.39 ± | 0.38 ± | 0.78 ± | 0.38 ± | 0.65 ± |
| 0.07 | 0.02 | 0.03 | 0.07 | 0.02 | 0.09 |

### Example 7—TfR Mediates PGRN Internalization in the Absence of Sortilin Binding and Promoted Cathepsin D Metabolism In Vitro

It has been reported that PGRN deficiency leads to Cathepsin D dysregulation in PGRN FTD (Gotzl et al, Acta Neuropathol, 2014). Thus, HEK293 cells were treated with PGRN-TfR fusion proteins with or without binding to human TfR or Sortilin receptor at 100 ng/mL for 24 hours. Mature Cathepsin D levels in cell lysate were evaluated by ELISA.

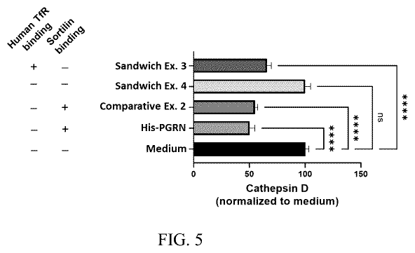

As shown in TABLE 12 and FIG. 5, TfR mediates PGRN internalization in the absence of Sortilin binding and promoted Cathepsin D metabolism in vitro. As shown in FIG. 5, Comparative Ex. 2 was able to shuttle PGRN into HEK293 cells. Comparative Ex. 2 can bind to sortilin, but not TfR. However, 8D3-PGRN-C90.43 could not shuttle PGRN into HEK293 cells because it cannot bind to sortilin or TfR. Unexpectedly, it was found that if the 8D3 Fab portion was replaced with H3.03, that the PGRN could be effectively shuttled into the HEK293 cells. One difference, as shown in FIG. 5, is that H3.03 can bind TfR, not sortilin. While not wishing to being bound by theory, it is believed that the data shown in TABLE 12 and FIG. 5 demonstrate that TfR binding can be an effective substitute for sortilin binding to transport PGRN across the BBB.

TABLE 12

| | | Comparative | Sandwich | Sandwich |
|---|---|---|---|---|
| | | Ex. 2 | Ex. 3 | Ex. 4 |
| | | 8D3-PGRN | 8D3-PGRN- | H3.03-PGRN- |
| Medium | His-PGRN | HEK | C90.43 | C90.43 |
| 100.00 ± | 50.39 ± | 55.08 ± | 100.03 ± | 66.00 ± |
| 2.52 | 3.65 | 1.96 | 4.00 | 3.00 |

### Example 8—Impact of Linker on Protein Quality

To assess the purification properties of the progranulin linker variants, 100 mL of tCHO supernatant containing the linker variant were purified using a 1-column process. Supernatant was applied to a 5 mL prepacked HiTrap PrismA resin column preequilibrated with 3 column volumes of 3 M Tris (pH 8.0). Column with loaded supernatant was subsequently washed with 2.5 column volumes of 20 mM Tris (pH 7), 2.5 column volumes of 20 mM Tris, 1 M NaCl (pH 7), and 2.5 column volumes of 20 mM Tris (pH 7). Fusion protein was eluted from PrismA resin by applying 5 column volumes of a mixed acid elution buffer (20 mM Acetic acid, 5 mM Citric acid). PrismA elution pool was neutralized to pH 5 with 1 M Tris (pH 8), centrifuged and applied to a 0.22-micron sterile filter. Protein concentration/yield was determined at A280 using the calculated extinction coefficient. Purity was evaluated with analytical size exclusion chromatography on a TSKgel UP—SW3000 column with isocratic elution in 50 mM Phosphate, 300 mM NaCl (pH 7) buffer.

As shown in TABLE 13, when the linker length was increased from (G₄Q)₁ to (G₄Q)₂ to (G₄Q)₃, the protein quality and purity increased in the case of both constructs.

TABLE 13
Impact of Linker Length on Protein Quality

| Construct | Linker Length | ProA Monomer % |
|---|---|---|
| H09-PGRN-C90.43 | (G4Q)₁ | 67 |
| | (G4Q)₂ | 74 |
| | (G4Q)₃ | 82 |
| H09-PGRNΔpGF-C90.43 | (G4Q)₁ | 88 |
| | (G4Q)₂ | 90 |
| | (G4Q)₃ | 92 |

SEQUENCE LISTING
Progranulin Sequences
SEQ ID NO. 1-Human Progranulin
TRCPDGQFCPVACCLDPGGASYSCCRPLLDKWPTTLSRHLGGPCQVDAHCSAGH
SCIFTVSGTSSCCPFPEAVACGDGHHCCPRGFHCSADGRSCFQRSGNNSVGAIQCP
DSQFECPDFSTCCVMVDGSWGCCPMPQASCCEDRVHCCPHGAFCDLVHTRCITP
TGTHPLAKKLPAQRTNRAVALSSSVMCPDARSRCPDGSTCCELPSGKYGCCPMP
NATCCSDHLHCCPQDTVCDLIQSKCLSKENATTDLLTKLPAHTVGDVKCDMEVS
CPDGYTCCRLQSGAWGCCPFTQAVCCEDHIHCCPAGFTCDTQKGTCEQGPHQVP
WMEKAPAHLSLPDPQALKRDVPCDNVSSCPSSDTCCQLTSGEWGCCPIPEAVCC
SDHQHCCPQGYTCVAEGQCQRGSEIVAGLEKMPARRASLSHPRDIGCDQHTSCP
VGQTCCPSLGGSWACCQLPHAVCCEDRQHCCPAGYTCNVKARSCEKEVVSAQP
ATFLARSPHVGVKDVECGEGHFCHDNQTCCRDNRQGWACCPYRQGVCCADRR
HCCPAGFRCAARGTKCLRREAPRWDAPLRDPALRQLL
SEQ ID NO. 2-Human Progranulin APGF
TPTGTHPLAKKLPAQRTNRAVALSSSVMCPDARSRCPDGSTCCELPSGKYGCCP
MPNATCCSDHLHCCPQDTVCDLIQSKCLSKENATTDLLTKLPAHTVGDVKCDME
VSCPDGYTCCRLQSGAWGCCPFTQAVCCEDHIHCCPAGFTCDTQKGTCEQGPHQ
VPWMEKAPAHLSLPDPQALKRDVPCDNVSSCPSSDTCCQLTSGEWGCCPIPEAV
CCSDHQHCCPQGYTCVAEGQCQRGSEIVAGLEKMPARRASLSHPRDIGCDQHTS
CPVGQTCCPSLGGSWACCQLPHAVCCEDRQHCCPAGYTCNVKARSCEKEVVSA
QPATFLARSPHVGVKDVECGEGHFCHDNQTCCRDNRQGWACCPYRQGVCCAD
RRHCCPAGFRCAARGTKCLRREAPRWDAPLRDPALRQLL
SEQ ID NO. 3-PGRNdesQLL
TRCPDGQFCPVACCLDPGGASYSCCRPLLDKWPTTLSRHLGGPCQVDAHCSAGH
SCIFTVSGTSSCCPFPEAVACGDGHHCCPRGFHCSADGRSCFQRSGNNSVGAIQCP
DSQFECPDFSTCCVMVDGSWGCCPMPQASCCEDRVHCCPHGAFCDLVHTRCITP
TGTHPLAKKLPAQRTNRAVALSSSVMCPDARSRCPDGSTCCELPSGKYGCCPMP
NATCCSDHLHCCPQDTVCDLIQSKCLSKENATTDLLTKLPAHTVGDVKCDMEVS
CPDGYTCCRLQSGAWGCCPFTQAVCCEDHIHCCPAGFTCDTQKGTCEQGPHQVP
WMEKAPAHLSLPDPQALKRDVPCDNVSSCPSSDTCCQLTSGEWGCCPIPEAVCC
SDHQHCCPQGYTCVAEGQCQRGSEIVAGLEKMPARRASLSHPRDIGCDQHTSCP
VGQTCCPSLGGSWACCQLPHAVCCEDRQHCCPAGYTCNVKARSCEKEVVSAQP
ATFLARSPHVGVKDVECGEGHFCHDNQTCCRDNRQGWACCPYRQGVCCADRR
HCCPAGFRCAARGTKCLRREAPRWDAPLQDPALR
SEQ ID NO. 4-Human Progranulin with QLL altered to PIL at amino
acid residue number 574-576
TRCPDGQFCPVACCLDPGGASYSCCRPLLDKWPTTLSRHLGGPCQVDAHCSAGH
SCIFTVSGTSSCCPFPEAVACGDGHHCCPRGFHCSADGRSCFQRSGNNSVGAIQCP
DSQFECPDFSTCCVMVDGSWGCCPMPQASCCEDRVHCCPHGAFCDLVHTRCITP
TGTHPLAKKLPAQRTNRAVALSSSVMCPDARSRCPDGSTCCELPSGKYGCCPMP
NATCCSDHLHCCPQDTVCDLIQSKCLSKENATTDLLTKLPAHTVGDVKCDMEVS
CPDGYTCCRLQSGAWGCCPFTQAVCCEDHIHCCPAGFTCDTQKGTCEQGPHQVP
WMEKAPAHLSLPDPQALKRDVPCDNVSSCPSSDTCCQLTSGEWGCCPIPEAVCC
SDHQHCCPQGYTCVAEGQCQRGSEIVAGLEKMPARRASLSHPRDIGCDQHTSCP
VGQTCCPSLGGSWACCQLPHAVCCEDRQHCCPAGYTCNVKARSCEKEVVSAQP
ATFLARSPHVGVKDVECGEGHFCHDNQTCCRDNRQGWACCPYRQGVCCADRR
HCCPAGFRCAARGTKCLRREAPRWDAPLRDPALRPIL
Peptide Linkers
SEQ ID NO. 5-(G4Q)₁
GGGGQ
SEQ ID NO. 6-(G4Q)₂
GGGGQGGGGQ
SEQ ID NO. 7-(G4Q)₃
GGGGQGGGGQGGGGQ
SEQ ID NO. 8-(G4Q)₄
GGGGQGGGGQGGGGQGGGGQ
SEQ ID NO. 9-(G4Q)₅
GGGGQGGGGQGGGGQGGGGQGGGGQ
SEQ ID NO. 10-(G4S)₁
GGGGS
SEQ ID NO. 11-(G4S)₂
GGGGSGGGGS
SEQ ID NO. 12-(G4S)₃
GGGGSGGGGSGGGGS
SEQ ID NO. 13-(G4S)₄
GGGGSGGGGSGGGGSGGGGS
SEQ ID NO. 14-(G4S)₅
GGGGSGGGGSGGGGSGGGGSGGGGS
Heavy Chains of Fab as TfR1 Binding Domain
SEQ ID NO. 15-H09 Fab Heavy Chain
EVQLVESGGGLVKPGGSLRLSCVASGFTFSSYSMNWVRQAPGKGLEWVSSISSSS
SYIYYADSVKGRFTISRDNAKNSLYLQMNSLRAEDTAVYYCARRHGYSNSDAFD
NWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSCDKTHT
SEQ ID NO. 16-H3.03 Fab Heavy Chain
EVQLVESGGGLVKPGGSLRLSCVASGFTFSSYSMNWVRQAPGKGLEWVSSISRSS
SYIYYADSVKGRFTISRDNAKNSLYLQMNSLRAEDTAVYYCARIHGYSNSDAFD
KWGQGTLVTVSSASTKGPCVFPLAPSSKSTSGGTAALGCLVKDYFPEPVTVSWN
SGALTSGVHTFPAVLQSSGLYSLSSVVTVPSSSLGTQTYICNVNHKPSNTKVDKK
VEPKSCDKTHT
SEQ ID NO. 17-8d3 Fab Heavy Chain
EVQLVESGGGLVQPGNSLTLSCVASGFTFSNYGMHWIRQAPKKGLEWIAMIYYD
SSKMNYADTVKGRFTISRDNSKNTLYLEMNSLRSEDTAMYYCAVPTSHYVVDV
WGQGVSVTVSSASTKGPCVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSCDKTHT
SEQ ID NO. 18-10E10 Fab Heavy Chain
QSLEESGGDLVKPEGSLTLTCTASGFSFSGSYWICWVRQAPGKGLEWIGCIYSTS
GGRTYYASWVKGRFTISKTSSTTVTLQMTSLTAADTATYFCARGDDSISDAYFDL
WGPGTLVTVSSASTKGPCVFPLAPCSRSTSESTAALGCLVKDYFPEPVTVSWNSG
ALTSGVHTFPAVLQSSGLYSLSSVVTVPSSSLGTKTYTCNVDHKPSNTKVDKRVE
SKYGPP
Light Chains of Fab as TfR1 Binding Domain
SEQ ID NO. 19-H09 Fab Light Chain
DIQMTQSPSAMSASVGDRVTITCRASQGISHYLVWFQQKPGKVPKRLIYAASSLQ
SGVPSRFSGSGSGTEFTLTISSLQPEDFATYYCLQHNSYPWTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO. 20-8D3 Fab Light Chain
DIQMTQSPASLSASLEEIVTITCQASQDIGNWLAWYQQKPGKSPQLLIYGATSLAD
GVPSRFSGSRSGTQFSLKISRVQVEDIGIYYCLQAYNTPWTFGGGTKLELKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO. 21-H3.03 Fab Light Chain
DIQMTQSPSAMSASVGDRVTITCRASQGISNYLAWFQQKPGKVPKRLIYAASSLQ
SGVPSRFSGSGSGTEFTLTISSLQPEDFATYYCLQHNSYPRTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO. 22-10E10 Fab Light Chain
ALDMTQTASPVSAAVGGTVTINCQSSQSVYNNNRLAWYQQKPGQPPKLLIYDAS
TLASGVPSRFKGSGSGTQFTLTISGVQSDDSATYYCQGTYFSSGWSWAFGGGTEV
VVKRADAAPTVSIFPPSSEQLTSGGASVVCFLNNFYPKDINVKWKIDGSERQNGV
LNSWTDQDSKDSTYSMSSTLTLTKDEYERHNSYTCEATHKTSTSPIVKSFNRNEC
V_{H}H as Albumin Binding Domain
SEQ ID NO. 23-C90.43 V_{H}H
EVQLLESGGGLVQPGGSLRLSCAASGRYIDETAVAWFRQAPGKGREFVAGIGGG
VDITYYADSVKGRFTISRDNSKNTLYLQMNSLRPEDTAVYYCGARPGRPLITSKV
ADLYPYWGQGTLVTVSSPP
SEQ ID NO. 24-C80.43 V_{H}H
EVQLLESGGGLVQPGGSLRLSCAASGRYIDETAVAWFRQAPGKGREFVAGIGGG
VDITYYADSVKGRFTISRDNSKNTLYLQMNSLRPEDTAVYYCAARPGRPLITSKV
ADLYPYWGQGTLVTVSSPP
Sandwich PGRN Payload
SEQ ID NO. 25-Heavy Chain of H09 Fab-PGRN-C90.43
EVQLVESGGGLVKPGGSLRLSCVASGFTFSSYSMNWVRQAPGKGLEWVSSISSSS
SYIYYADSVKGRFTISRDNAKNSLYLQMNSLRAEDTAVYYCARRHGYSNSDAFD
NWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSCDKTHTGGGGQGGGGQGGGGQTRCPDGQFCPVACCLDPGGASYSCCRPL
LDKWPTTLSRHLGGPCQVDAHCSAGHSCIFTVSGTSSCCPFPEAVACGDGHHCCP
RGFHCSADGRSCFQRSGNNSVGAIQCPDSQFECPDFSTCCVMVDGSWGCCPMPQ
ASCCEDRVHCCPHGAFCDLVHTRCITPTGTHPLAKKLPAQRTNRAVALSSSVMC
PDARSRCPDGSTCCELPSGKYGCCPMPNATCCSDHLHCCPQDTVCDLIQSKCLSK
ENATTDLLTKLPAHTVGDVKCDMEVSCPDGYTCCRLQSGAWGCCPFTQAVCCE
DHIHCCPAGFTCDTQKGTCEQGPHQVPWMEKAPAHLSLPDPQALKRDVPCDNV
SSCPSSDTCCQLTSGEWGCCPIPEAVCCSDHQHCCPQGYTCVAEGQCQRGSEIVA
GLEKMPARRASLSHPRDIGCDQHTSCPVGQTCCPSLGGSWACCQLPHAVCCEDR
QHCCPAGYTCNVKARSCEKEVVSAQPATFLARSPHVGVKDVECGEGHFCHDNQ
TCCRDNRQGWACCPYRQGVCCADRRHCCPAGFRCAARGTKCLRREAPRWDAP
LRDPALRQLLGGGGQGGGGQGGGGQEVQLLESGGGLVQPGGSLRLSCAASGRY
IDETAVAWFRQAPGKGREFVAGIGGGVDITYYADSVKGRFTISRDNSKNTLYLQ
MNSLRPEDTAVYYCGARPGRPLITSKVADLYPYWGQGTLVTVSSPP
SEQ ID NO. 26-H09 Fab-PGRNApGF-C90.43 Heavy Chain
EVQLVESGGGLVKPGGSLRLSCVASGFTFSSYSMNWVRQAPGKGLEWVSSISSSS
SYIYYADSVKGRFTISRDNAKNSLYLQMNSLRAEDTAVYYCARRHGYSNSDAFD
NWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSCDKTGGGGQGGGGQGGGGQTPTGTHPLAKKLPAQRTNRAVALSSSVMCP
DARSRCPDGSTCCELPSGKYGCCPMPNATCCSDHLHCCPQDTVCDLIQSKCLSKE
NATTDLLTKLPAHTVGDVKCDMEVSCPDGYTCCRLQSGAWGCCPFTQAVCCED
HIHCCPAGFTCDTQKGTCEQGPHQVPWMEKAPAHLSLPDPQALKRDVPCDNVSS
CPSSDTCCQLTSGEWGCCPIPEAVCCSDHQHCCPQGYTCVAEGQCQRGSEIVAGL
EKMPARRASLSHPRDIGCDQHTSCPVGQTCCPSLGGSWACCQLPHAVCCEDRQH
CCPAGYTCNVKARSCEKEVVSAQPATFLARSPHVGVKDVECGEGHFCHDNQTC
CRDNRQGWACCPYRQGVCCADRRHCCPAGFRCAARGTKCLRREAPRWDAPLR
DPALRQLLGGGGQGGGGQGGGGQEVQLLESGGGLVQPGGSLRLSCAASGRYID
ETAVAWFRQAPGKGREFVAGIGGGVDITYYADSVKGRFTISRDNSKNTLYLQMN
SLRPEDTAVYYCGARPGRPLITSKVADLYPYWGQGTLVTVSSPP
SEQ ID NO. 27-8D3-PGRN-C90.43 Heavy Chain
EVQLVESGGGLVQPGNSLTLSCVASGFTFSNYGMHWIRQAPKKGLEWIAMIYYD
SSKMNYADTVKGRFTISRDNSKNTLYLEMNSLRSEDTAMYYCAVPTSHYVVDV
WGQGVSVTVSSASTKGPCVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSCDKTHTGGGGQGGGGQGGGGQTRCPDGQFCPVACCLDPGGASYSCCRPL
LDKWPTTLSRHLGGPCQVDAHCSAGHSCIFTVSGTSSCCPFPEAVACGDGHHCCP
RGFHCSADGRSCFQRSGNNSVGAIQCPDSQFECPDFSTCCVMVDGSWGCCPMPQ
ASCCEDRVHCCPHGAFCDLVHTRCITPTGTHPLAKKLPAQRTNRAVALSSSVMC
PDARSRCPDGSTCCELPSGKYGCCPMPNATCCSDHLHCCPQDTVCDLIQSKCLSK
ENATTDLLTKLPAHTVGDVKCDMEVSCPDGYTCCRLQSGAWGCCPFTQAVCCE
DHIHCCPAGFTCDTQKGTCEQGPHQVPWMEKAPAHLSLPDPQALKRDVPCDNV
SSCPSSDTCCQLTSGEWGCCPIPEAVCCSDHQHCCPQGYTCVAEGQCQRGSEIVA
GLEKMPARRASLSHPRDIGCDQHTSCPVGQTCCPSLGGSWACCQLPHAVCCEDR
QHCCPAGYTCNVKARSCEKEVVSAQPATFLARSPHVGVKDVECGEGHFCHDNQ
TCCRDNRQGWACCPYRQGVCCADRRHCCPAGFRCAARGTKCLRREAPRWDAP
LRDPALRQLLGGGGQGGGGQGGGGQEVQLLESGGGLVQPGGSLRLSCAASGRY
IDETAVAWFRQAPGKGREFVAGIGGGVDITYYADSVKGRFTISRDNSKNTLYLQ
MNSLRPEDTAVYYCGARPGRPLITSKVADLYPYWGQGTLVTVSSPP
SEQ ID NO. 28-H3.03-PGRN-C90.43 Heavy Chain
EVQLVESGGGLVKPGGSLRLSCVASGFTFSSYSMNWVRQAPGKGLEWVSSISRSS
SYIYYADSVKGRFTISRDNAKNSLYLQMNSLRAEDTAVYYCARIHGYSNSDAFD
KWGQGTLVTVSSASTKGPCVFPLAPSSKSTSGGTAALGCLVKDYFPEPVTVSWN
SGALTSGVHTFPAVLQSSGLYSLSSVVTVPSSSLGTQTYICNVNHKPSNTKVDKK
VEPKSCDKTHTGGGGQGGGGQGGGGQTRCPDGQFCPVACCLDPGGASYSCCRP
LLDKWPTTLSRHLGGPCQVDAHCSAGHSCIFTVSGTSSCCPFPEAVACGDGHHCC
PRGFHCSADGRSCFQRSGNNSVGAIQCPDSQFECPDFSTCCVMVDGSWGCCPMP
QASCCEDRVHCCPHGAFCDLVHTRCITPTGTHPLAKKLPAQRTNRAVALSSSVM
CPDARSRCPDGSTCCELPSGKYGCCPMPNATCCSDHLHCCPQDTVCDLIQSKCLS
KENATTDLLTKLPAHTVGDVKCDMEVSCPDGYTCCRLQSGAWGCCPFTQAVCC
EDHIHCCPAGFTCDTQKGTCEQGPHQVPWMEKAPAHLSLPDPQALKRDVPCDN
VSSCPSSDTCCQLTSGEWGCCPIPEAVCCSDHQHCCPQGYTCVAEGQCQRGSEIV
AGLEKMPARRASLSHPRDIGCDQHTSCPVGQTCCPSLGGSWACCQLPHAVCCED
RQHCCPAGYTCNVKARSCEKEVVSAQPATFLARSPHVGVKDVECGEGHFCHDN
QTCCRDNRQGWACCPYRQGVCCADRRHCCPAGFRCAARGTKCLRREAPRWDA
PLRDPALRQLLGGGGQGGGGQGGGGQEVQLLESGGGLVQPGGSLRLSCAASGR
YIDETAVAWFRQAPGKGREFVAGIGGGVDITYYADSVKGRFTISRDNSKNTLYL
QMNSLRPEDTAVYYCGARPGRPLITSKVADLYPYWGQGTLVTVSSPP
Kappa PGRN Payload
SEQ ID NO. 29-H09-C90.43 Heavy Chain
EVQLVESGGGLVKPGGSLRLSCVASGFTFSSYSMNWVRQAPGKGLEWVSSISSSS
SYIYYADSVKGRFTISRDNAKNSLYLQMNSLRAEDTAVYYCARRHGYSNSDAFD
NWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSCDKTGGGGQGGGGQGGGGQGGGGQGGGGQEVQLLESGGGLVQPGGSLR
LSCAASGRYIDETAVAWFRQAPGKGREFVAGIGGGVDITYYADSVKGRFTISRDN
SKNTLYLQMNSLRPEDTAVYYCGARPGRPLITSKVADLYPYWGQGTLVTVSSPP
SEQ ID NO. 30-H09 PGRNdesQLL Light Chain
DIQMTQSPSAMSASVGDRVTITCRASQGISHYLVWFQQKPGKVPKRLIYAASSLQ
SGVPSRFSGSGSGTEFTLTISSLQPEDFATYYCLQHNSYPWTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECGGGGQG
GGGQGGGGQGGGGQGGGGQTRCPDGQFCPVACCLDPGGASYSCCRPLLDKWP
TTLSRHLGGPCQVDAHCSAGHSCIFTVSGTSSCCPFPEAVACGDGHHCCPRGFHC
SADGRSCFQRSGNNSVGAIQCPDSQFECPDFSTCCVMVDGSWGCCPMPQASCCE
DRVHCCPHGAFCDLVHTRCITPTGTHPLAKKLPAQRTNRAVALSSSVMCPDARS
RCPDGSTCCELPSGKYGCCPMPNATCCSDHLHCCPQDTVCDLIQSKCLSKENATT
DLLTKLPAHTVGDVKCDMEVSCPDGYTCCRLQSGAWGCCPFTQAVCCEDHIHC
CPAGFTCDTQKGTCEQGPHQVPWMEKAPAHLSLPDPQALKRDVPCDNVSSCPSS
DTCCQLTSGEWGCCPIPEAVCCSDHQHCCPQGYTCVAEGQCQRGSEIVAGLEKM
PARRASLSHPRDIGCDQHTSCPVGQTCCPSLGGSWACCQLPHAVCCEDRQHCCP
AGYTCNVKARSCEKEVVSAQPATFLARSPHVGVKDVECGEGHFCHDNQTCCRD
NRQGWACCPYRQGVCCADRRHCCPAGFRCAARGTKCLRREAPRWDAPLQDPA
LR
SEQ ID NO. 31-H09 PGRNApGF desQLL Light Chain
DIQMTQSPSAMSASVGDRVTITCRASQGISHYLVWFQQKPGKVPKRLIYAASSLQ
SGVPSRFSGSGSGTEFTLTISSLQPEDFATYYCLQHNSYPWTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECGGGGQG
GGGQGGGGQGGGGQGGGGQTPTGTHPLAKKLPAQRTNRAVALSSSVMCPDAR
SRCPDGSTCCELPSGKYGCCPMPNATCCSDHLHCCPQDTVCDLIQSKCLSKENAT
TDLLTKLPAHTVGDVKCDMEVSCPDGYTCCRLQSGAWGCCPFTQAVCCEDHIH
CCPAGFTCDTQKGTCEQGPHQVPWMEKAPAHLSLPDPQALKRDVPCDNVSSCPS
SDTCCQLTSGEWGCCPIPEAVCCSDHQHCCPQGYTCVAEGQCQRGSEIVAGLEK
MPARRASLSHPRDIGCDQHTSCPVGQTCCPSLGGSWACCQLPHAVCCEDRQHCC
PAGYTCNVKARSCEKEVVSAQPATFLARSPHVGVKDVECGEGHFCHDNQTCCR
DNRQGWACCPYRQGVCCADRRHCCPAGFRCAARGTKCLRREAPRWDAPLRDP
ALR
SEQ ID NO. 32-H09 PGRN Light Chain
DIQMTQSPSAMSASVGDRVTITCRASQGISHYLVWFQQKPGKVPKRLIYAASSLQ
SGVPSRFSGSGSGTEFTLTISSLQPEDFATYYCLQHNSYPWTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECGGGGQG
GGGQGGGGQGGGGQGGGGQTRCPDGQFCPVACCLDPGGASYSCCRPLLDKWP
TTLSRHLGGPCQVDAHCSAGHSCIFTVSGTSSCCPFPEAVACGDGHHCCPRGFHC
SADGRSCFQRSGNNSVGAIQCPDSQFECPDFSTCCVMVDGSWGCCPMPQASCCE
DRVHCCPHGAFCDLVHTRCITPTGTHPLAKKLPAQRTNRAVALSSSVMCPDARS
RCPDGSTCCELPSGKYGCCPMPNATCCSDHLHCCPQDTVCDLIQSKCLSKENATT
DLLTKLPAHTVGDVKCDMEVSCPDGYTCCRLQSGAWGCCPFTQAVCCEDHIHC
CPAGFTCDTQKGTCEQGPHQVPWMEKAPAHLSLPDPQALKRDVPCDNVSSCPSS
DTCCQLTSGEWGCCPIPEAVCCSDHQHCCPQGYTCVAEGQCQRGSEIVAGLEKM
PARRASLSHPRDIGCDQHTSCPVGQTCCPSLGGSWACCQLPHAVCCEDRQHCCP
AGYTCNVKARSCEKEVVSAQPATFLARSPHVGVKDVECGEGHFCHDNQTCCRD
NRQGWACCPYRQGVCCADRRHCCPAGFRCAARGTKCLRREAPRWDAPLQDPA
LRQLL
SEQ ID NO. 33-H09 PGRNApGF Light Chain
DIQMTQSPSAMSASVGDRVTITCRASQGISHYLVWFQQKPGKVPKRLIYAASSLQ
SGVPSRFSGSGSGTEFTLTISSLQPEDFATYYCLQHNSYPWTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECGGGGQG
GGGQGGGGQGGGGQGGGGQTPTGTHPLAKKLPAQRTNRAVALSSSVMCPDAR
SRCPDGSTCCELPSGKYGCCPMPNATCCSDHLHCCPQDTVCDLIQSKCLSKENAT
TDLLTKLPAHTVGDVKCDMEVSCPDGYTCCRLQSGAWGCCPFTQAVCCEDHIH
CCPAGFTCDTQKGTCEQGPHQVPWMEKAPAHLSLPDPQALKRDVPCDNVSSCPS
SDTCCQLTSGEWGCCPIPEAVCCSDHQHCCPQGYTCVAEGQCQRGSEIVAGLEK
MPARRASLSHPRDIGCDQHTSCPVGQTCCPSLGGSWACCQLPHAVCCEDRQHCC
PAGYTCNVKARSCEKEVVSAQPATFLARSPHVGVKDVECGEGHFCHDNQTCCR
DNRQGWACCPYRQGVCCADRRHCCPAGFRCAARGTKCLRREAPRWDAPLRDP
ALRQLL
SEQ ID NO. 34-10E10-C80.43 Heavy Chain
QSLEESGGDLVKPEGSLTLTCTASGFSFSGSYWICWVRQAPGKGLEWIGCIYSTS
GGRTYYASWVKGRFTISKTSSTTVTLQMTSLTAADTATYFCARGDDSISDAYFDL
WGPGTLVTVSSASTKGPCVFPLAPCSRSTSESTAALGCLVKDYFPEPVTVSWNSG
ALTSGVHTFPAVLQSSGLYSLSSVVTVPSSSLGTKTYTCNVDHKPSNTKVDKRVE
SKYGPPGGGGQGGGGQGGGGQGGGGQGGGGQEVQLLESGGGLVQPGGSLRLS
CAASGRYIDETAVAWFRQAPGKGREFVAGIGGGVDITYYADSVKGRFTISRDNS
KNTLYLQMNSLRPEDTAVYYCAARPGRPLITSKVADLYPYWGQGTLVTVSSPP
SEQ ID NO. 35-10E10 PGRN Light Chain
ALDMTQTASPVSAAVGGTVTINCQSSQSVYNNNRLAWYQQKPGQPPKLLIYDAS
TLASGVPSRFKGSGSGTQFTLTISGVQSDDSATYYCQGTYFSSGWSWAFGGGTEV
VVKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGE
CGGGGQGGGGQGGGGQGGGGQGGGGQTRCPDGQFCPVACCLDPGGASYSCCR
PLLDKWPTTLSRHLGGPCQVDAHCSAGHSCIFTVSGTSSCCPFPEAVACGDGHHC
CPRGFHCSADGRSCFQRSGNNSVGAIQCPDSQFECPDFSTCCVMVDGSWGCCPM
PQASCCEDRVHCCPHGAFCDLVHTRCITPTGTHPLAKKLPAQRTNRAVALSSSV
MCPDARSRCPDGSTCCELPSGKYGCCPMPNATCCSDHLHCCPQDTVCDLIQSKC
LSKENATTDLLTKLPAHTVGDVKCDMEVSCPDGYTCCRLQSGAWGCCPFTQAV
CCEDHIHCCPAGFTCDTQKGTCEQGPHQVPWMEKAPAHLSLPDPQALKRDVPCD
NVSSCPSSDTCCQLTSGEWGCCPIPEAVCCSDHQHCCPQGYTCVAEGQCQRGSEI
VAGLEKMPARRASLSHPRDIGCDQHTSCPVGQTCCPSLGGSWACCQLPHAVCCE
DRQHCCPAGYTCNVKARSCEKEVVSAQPATFLARSPHVGVKDVECGEGHFCHD
NQTCCRDNRQGWACCPYRQGVCCADRRHCCPAGFRCAARGTKCLRREAPRWD
APLRDPALRQLL
SEQ ID NO. 36-10E10 PGRNApGF Light Chain
ALDMTQTASPVSAAVGGTVTINCQSSQSVYNNNRLAWYQQKPGQPPKLLIYDAS
TLASGVPSRFKGSGSGTQFTLTISGVQSDDSATYYCQGTYFSSGWSWAFGGGTEV
VVKRADAAPTVSIFPPSSEQLTSGGASVVCFLNNFYPKDINVKWKIDGSERQNGV
LNSWTDQDSKDSTYSMSSTLTLTKDEYERHNSYTCEATHKTSTSPIVKSFNRNEC
GGGGQGGGGQGGGGQGGGGQGGGGQTPTGTHPLAKKLPAQRTNRAVALSSSV
MCPDARSRCPDGSTCCELPSGKYGCCPMPNATCCSDHLHCCPQDTVCDLIQSKC
LSKENATTDLLTKLPAHTVGDVKCDMEVSCPDGYTCCRLQSGAWGCCPFTQAV
CCEDHIHCCPAGFTCDTQKGTCEQGPHQVPWMEKAPAHLSLPDPQALKRDVPCD
NVSSCPSSDTCCQLTSGEWGCCPIPEAVCCSDHQHCCPQGYTCVAEGQCQRGSEI
VAGLEKMPARRASLSHPRDIGCDQHTSCPVGQTCCPSLGGSWACCQLPHAVCCE
DRQHCCPAGYTCNVKARSCEKEVVSAQPATFLARSPHVGVKDVECGEGHFCHD
NQTCCRDNRQGWACCPYRQGVCCADRRHCCPAGFRCAARGTKCLRREAPRWD
APLRDPALRQLL
SEQ ID NO. 37-10E10 R32W (300nM) PGRN Light Chain
ALDMTQTASPVSAAVGGTVTINCQSSQSVYNNNWLAWYQQKPGQPPKLLIYDA
STLASGVPSRFKGSGSGTQFTLTISGVQSDDSATYYCQGTYFSSGWSWAFGGGTE
VVVKRADAAPTVSIFPPSSEQLTSGGASVVCFLNNFYPKDINVKWKIDGSERQNG
VLNSWTDQDSKDSTYSMSSTLTLTKDEYERHNSYTCEATHKTSTSPIVKSFNRNE
CGGGGQGGGGQGGGGQGGGGQGGGGQTRCPDGQFCPVACCLDPGGASYSCCR
PLLDKWPTTLSRHLGGPCQVDAHCSAGHSCIFTVSGTSSCCPFPEAVACGDGHHC
CPRGFHCSADGRSCFQRSGNNSVGAIQCPDSQFECPDFSTCCVMVDGSWGCCPM
PQASCCEDRVHCCPHGAFCDLVHTRCITPTGTHPLAKKLPAQRTNRAVALSSSV
MCPDARSRCPDGSTCCELPSGKYGCCPMPNATCCSDHLHCCPQDTVCDLIQSKC
LSKENATTDLLTKLPAHTVGDVKCDMEVSCPDGYTCCRLQSGAWGCCPFTQAV
CCEDHIHCCPAGFTCDTQKGTCEQGPHQVPWMEKAPAHLSLPDPQALKRDVPCD
NVSSCPSSDTCCQLTSGEWGCCPIPEAVCCSDHQHCCPQGYTCVAEGQCQRGSEI
VAGLEKMPARRASLSHPRDIGCDQHTSCPVGQTCCPSLGGSWACCQLPHAVCCE
DRQHCCPAGYTCNVKARSCEKEVVSAQPATFLARSPHVGVKDVECGEGHFCHD
NQTCCRDNRQGWACCPYRQGVCCADRRHCCPAGFRCAARGTKCLRREAPRWD
APLRDPALRQLL
SEQ ID NO. 38-10E10 T52G PGRN Light Chain (1000 nM)
AIDMTQTASPVSAAVGGTVTINCQSSQSVYNNNRLAWYQQKPGQPPKLLIYGAS
TLASGVPSRFKGSGSGTQFTLTISGVQCDDSATYYCQGTYFSSGWSWAFGGGTE
VVVKRADAAPTVSIFPPSSEQLTSGGASVVCFLNNFYPKDINVKWKIDGSERQNG
VLNSWTDQDSKDSTYSMSSTLTLTKDEYERHNSYTCEATHKTSTSPIVKSFNRNE
CGGGGQGGGGQGGGGQGGGGQGGGGQTRCPDGQFCPVACCLDPGGASYSCCR
PLLDKWPTTLSRHLGGPCQVDAHCSAGHSCIFTVSGTSSCCPFPEAVACGDGHHC
CPRGFHCSADGRSCFQRSGNNSVGAIQCPDSQFECPDFSTCCVMVDGSWGCCPM
PQASCCEDRVHCCPHGAFCDLVHTRCITPTGTHPLAKKLPAQRTNRAVALSSSV
MCPDARSRCPDGSTCCELPSGKYGCCPMPNATCCSDHLHCCPQDTVCDLIQSKC
LSKENATTDLLTKLPAHTVGDVKCDMEVSCPDGYTCCRLQSGAWGCCPFTQAV
CCEDHIHCCPAGFTCDTQKGTCEQGPHQVPWMEKAPAHLSLPDPQALKRDVPCD
NVSSCPSSDTCCQLTSGEWGCCPIPEAVCCSDHQHCCPQGYTCVAEGQCQRGSEI
VAGLEKMPARRASLSHPRDIGCDQHTSCPVGQTCCPSLGGSWACCQLPHAVCCE
DRQHCCPAGYTCNVKARSCEKEVVSAQPATFLARSPHVGVKDVECGEGHFCHD
NQTCCRDNRQGWACCPYRQGVCCADRRHCCPAGFRCAARGTKCLRREAPRWD
APLRDPALRQLL
Knob and Hole
SEQ ID NO. 39-scFv 8D3-hIgG1-LALA knob
EVQLVESGGGLVQPGNSLTLSCVASGFTFSNYGMHWIRQAPKKCLEWIAMIYYD
SSKMNYADTVKGRFTISRDNSKNTLYLEMNSLRSEDTAMYYCAVPTSHYVVDV
WGQGVSVTVSSGGGGSGGGGSGGGGSGGGGSGGGGSDIQMTQSPASLSASLEEI
VTITCQASQDIGNWLAWYQQKPGKSPQLLIYGATSLADGVPSRFSGSRSGTQFSL
KISRVQVEDIGIYYCLQAYNTPWTFGCGTKLELKGGGGQGGGGQGGGGQDKTH
TCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPI
EKTISKAKGQPREPQVYTLPPSRDELTKNQVSLWCLVKGFYPSDIAVEWESNGQP
ENNYKTTPPVLDSDGSFFLYSKLTVDKSRWQQGNVFSCSVMHEALHNHYTQKSL
SLSPGK
SEQ ID NO. 40-hIgG1 LALA hole-PGRN
DKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLSCAVKGFYPSDIAVEWES
NGQPENNYKTTPPVLDSDGSFFLVSKLTVDKSRWQQGNVFSCSVMHEALHNHY
TQKSLSLSPGKGGGGSGGGGSTRCPDGQFCPVACCLDPGGASYSCCRPLLDKWP
TTLSRHLGGPCQVDAHCSAGHSCIFTVSGTSSCCPFPEAVACGDGHHCCPRGFHC
SADGRSCFQRSGNNSVGAIQCPDSQFECPDFSTCCVMVDGSWGCCPMPQASCCE
DRVHCCPHGAFCDLVHTRCITPTGTHPLAKKLPAQRTNRAVALSSSVMCPDARS
RCPDGSTCCELPSGKYGCCPMPNATCCSDHLHCCPQDTVCDLIQSKCLSKENATT
DLLTKLPAHTVGDVKCDMEVSCPDGYTCCRLQSGAWGCCPFTQAVCCEDHIHC
CPAGFTCDTQKGTCEQGPHQVPWMEKAPAHLSLPDPQALKRDVPCDNVSSCPSS
DTCCQLTSGEWGCCPIPEAVCCSDHQHCCPQGYTCVAEGQCQRGSEIVAGLEKM
PARRASLSHPRDIGCDQHTSCPVGQTCCPSLGGSWACCQLPHAVCCEDRQHCCP
AGYTCNVKARSCEKEVVSAQPATFLARSPHVGVKDVECGEGHFCHDNQTCCRD
NRQGWACCPYRQGVCCADRRHCCPAGFRCAARGTKCLRREAPRWDAPLRDPA
LRQLL
SEQ ID NO. 41-3F5 Light Chain
AEVVMTQTPSSVSAAVGGTVTIKCQASQNINSWLSWYQQKPGQRPKLLIYSAST
LASGVPSRFEGSGSGTEYTLTISDLECDDAATYYCQSSYGSSYDFGGGTEVVVKG
DLVAPTVLIFPPAADQVATGTVTIVCVANKYFPDVTVTWEVDGTTQTTGIENSKT
PQNSADCTYNLSSTLTLTSTQYNSHKEYTCKVTQGTTSVVQSFNRGDC
SEQ ID NO. 42-3F5 hIgG1 LALA knob
QVQLVQSGAEVKKPGSSVKVSCTASGFSFSSGYWICWVRQAPGQGLEWMGCIH
SVRSHMTYYAQKFQGRVTITADKSTSTAYMELSSLRSEDTAVYYCARDASGVW
NYFTLWGQGTLVTVSSASTKGPCVFPLAPCSRSTSESTAALGCLVKDYFPEPVTV
SWNSGALTSGVHTFPAVLQSSGLYSLSSVVTVPSSSLGTKTYTCNVDHKPSNTKV
DKRVESKYCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYKCKVS
NKALPAPIEKTISKAKGQPREPQVYTLPPSRDELTKNQVSLWCLVKGFYPSDIAVE
WESNGQPENNYKTTPPVLDSDGSFFLYSKLTVDKSRWQQGNVFSCSVMHEALH
NHYTQKSLSLSPGK
SEQ ID NO. 43-stump hlgG1 TV LALA knob
TRCPDGQFCPVACCLDPGGASYSCCRPLLDKWPTTLSRHLGGPCQVDAHCSAGH
SCIFTVSGTSSCCPFPEAVACGDGHHCCPRGFHCSADGRSCFQRSGNNSVGAIQCP
DSQFECPDFSTCCVMVDGSWGCCPMPQASCCEDRVHCCPHGAFCDLVHTRCITP
TGTHPLAKKLPAQRTNRAVALSSSVMCPDARSRCPDGSTCCELPSGKYGCCPMP
NATCCSDHLHCCPQDTVCDLIQSKCLSKENATTDLLTKLPAHTVGDVKCDMEVS
CPDGYTCCRLQSGAWGCCPFTQAVCCEDHIHCCPAGFTCDTQKGTCEQGPHQVP
WMEKAPAHLSLPDPQALKRDVPCDNVSSCPSSDTCCQLTSGEWGCCPIPEAVCC
SDHQHCCPQGYTCVAEGQCQRGSEIVAGLEKMPARRASLSHPRDIGCDQHTSCP
VGQTCCPSLGGSWACCQLPHAVCCEDRQHCCPAGYTCNVKARSCEKEVVSAQP
ATFLARSPHVGVKDVECGEGHFCHDNQTCCRDNRQGWACCPYRQGVCCADRR
HCCPAGFRCAARGTKCLRREAPRWDAPLRDPALRQLLGGGGSGGGGSDKTHTC
PPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEK
TISKAKGQPREPQVYTLPPSRDELTKNQVSLSCAVKGFYPSDIAVEWESNGQPEN
NYKTTPPVLDSDGSFFLVSKLTVDKSRWQQGNVFSCSVMHEALHNHYTQKSLSL
SPGK
SEQ ID NO. 44-stump hIgG1 LALA knob
DKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
PAPIEKTISKAKGQPREPQVYTLPPSRDELTKNQVSLWCLVKGFYPSDIAVEWESN
GQPENNYKTTPPVLDSDGSFFLYSKLTVDKSRWQQGNVFSCSVMHEALHNHYT
QKSLSLSPGK
TfR1 Binding Domain Complementary Determining Regions
SEQ ID NO. 45-Heavy Chain VH CDR1-North
VASGFTFSSYSMN
SEQ ID NO. 46-Heavy Chain VH CDR2-Kabat
SISSSSSYIYYADSVKG
SEQ ID NO. 47-Heavy Chain VH CDR3-North
ARRHGYSNSDAFDN
SEQ ID NO. 48-Light Chain Kappa CDR1-North
RASQGISHYLV
SEQ ID NO. 49-Light Chain Kappa CDR2-North
YAASSLQS
SEQ ID NO. 50-Light Chain Kappa CDR3-North
LQHNSYPWT
Albumin Binding Domain Complementary Determining Regions
SEQ ID NO. 51-C90.43 CDR1
AASGRYIDETAVA
SEQ ID NO. 52-C90.43 CDR2
GIGGGVDITYYADSVKG
SEQ ID NO. 53-C90.43 CDR3
GARPGRPLITSKVADLYPY
TfR 1 Binding Domain Variable Heavy (V_{H})
SEQ ID NO. 54-H09 Fab V_{H} (HCVR)
EVQLVESGGGLVKPGGSLRLSCVASGFTFSSYSMNWVRQAPGKGLEWVSSISSSS
SYIYYADSVKGRFTISRDNAKNSLYLQMNSLRAEDTAVYYCARRHGYSNSDAFD
NWGQGTLVTVSS
Other Sequences
SEQ ID NO. 55-His-Tagged PGRN
HHHHHHGGGGQGGGGQGGGGQENLYFQSGGGGQTRCPDGQFCPVACCLDPGG
ASYSCCRPLLDKWPTTLSRHLGGPCQVDAHCSAGHSCIFTVSGTSSCCPFPEAVA
CGDGHHCCPRGFHCSADGRSCFQRSGNNSVGAIQCPDSQFECPDFSTCCVMVDG
SWGCCPMPQASCCEDRVHCCPHGAFCDLVHTRCITPTGTHPLAKKLPAQRTNRA
VALSSSVMCPDARSRCPDGSTCCELPSGKYGCCPMPNATCCSDHLHCCPQDTVC
DLIQSKCLSKENATTDLLTKLPAHTVGDVKCDMEVSCPDGYTCCRLQSGAWGC
CPFTQAVCCEDHIHCCPAGFTCDTQKGTCEQGPHQVPWMEKAPAHLSLPDPQAL
KRDVPCDNVSSCPSSDTCCQLTSGEWGCCPIPEAVCCSDHQHCCPQGYTCVAEG
QCQRGSEIVAGLEKMPARRASLSHPRDIGCDQHTSCPVGQTCCPSLGGSWACCQ
LPHAVCCEDRQHCCPAGYTCNVKARSCEKEVVSAQPATFLARSPHVGVKDVEC
GEGHFCHDNQTCCRDNRQGWACCPYRQGVCCADRRHCCPAGFRCAARGTKCL
RREAPRWDAPLRDPALRQLL
Additional Definitions of the TfR1 Binding Domain Complementary
Determining Regions
SEQ ID NO. 56 -Heavy Chain VH CDR2-North
SISSSSSYIY
SEQ ID NO. 57-Heavy Chain VH CDR1-Kabat
SYSMN
SEQ ID NO. 58-Heavy Chain VH CDR3-Kabat
RHGYSNSDAFDN
SEQ ID NO. 59-Light Chain Kappa CDR1-Kabat
RASQGISHYLV
SEQ ID NO. 60-Light Chain Kappa CDR2-Kabat
AASSLQS
SEQ ID NO. 61-Light Chain Kappa CDR3-Kabat
LQHNSYPWT
SEQ ID NO. 62-Heavy Chain VH CDR1-IMGT
GFTFSSYS
SEQ ID NO. 63-Heavy Chain VH CDR2-IMGT
ISSSSSYI
SEQ ID NO. 64-Heavy Chain VH CDR3-IMGT
ARRHGYSNSDAFDN
SEQ ID NO. 65-Light Chain Kappa CDR1-IMGT
QGISHY
SEQ ID NO. 66-Light Chain Kappa CDR2-IMGT
AAS
SEQ ID NO. 67-Light Chain Kappa CDR3-IMGT
LQHNSYPWT
SEQ ID NO. 68-Heavy Chain VH CDR1-Chothia
GFTFSSY
SEQ ID NO. 69 Heavy Chain VH CDR2-Chothia
SSSSSY
SEQ ID NO. 70-Heavy Chain VH CDR3-Chothia
RHGYSNSDAFDN
SEQ ID NO. 71-Light Chain Kappa CDR1-Chothia
RASQGISHYLV
SEQ ID NO. 72-Light Chain Kappa CDR2-Chothia
AASSLQS
SEQ ID NO. 73-Light Chain Kappa CDR3-Chothia
LQHNSYPWT

What is claimed is:

1. A compound comprising:
(a) a progranulin domain; and
(b) a transferrin receptor 1 (TfR1) binding domain, wherein the TfR1 binding domain comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein
the amino acid sequence of HCDR1 is SEQ ID NO. 45,
the amino acid sequence of HCDR2 is SEQ ID NO. 46,
the amino acid sequence of HCDR3 is SEQ ID NO. 47,
the amino acid sequence of LCDR1 is SEQ ID NO. 48,
the amino acid sequence of LCDR2 is SEQ ID NO. 49, and
the amino acid sequence of LCDR3 is SEQ ID NO. 50.

2. The compound of claim 1, wherein the compound further comprises an albumin binding domain, wherein the albumin binding domain the comprises complementarity determining regions (CDR) CDR1, CDR2, and CDR3, wherein
the amino acid sequence of CDR1 is SEQ ID NO: 51,
the amino acid sequence of CDR2 is SEQ ID NO: 52, and
the amino acid sequence of CDR3 is SEQ ID NO: 53.

3. The compound of claim 2, wherein the TfR1 binding domain is linked to the progranulin domain with a first linker and the albumin binding domain is linked to the progranulin domain or the TfR1 binding domain with a second linker.

4. The compound of claim 3, wherein the compound is of the formula:
Y-L₁-X-L₂-Z or Z-L₂-Y-L₁-X and
wherein X is the progranulin domain,
Y is the TfR1 binding domain,
Z is the albumin binding domain,
L₁ is the first linker, and
L₂ is the second linker.

5. The compound of claim 4, wherein the compound is of the formula:
Y-L₁-X-L₂-Z, and
wherein the TfR1 binding domain is linked to the progranulin domain at an N-terminus of the progranulin domain and the albumin binding domain is linked to the progranulin at a C-terminus of the progranulin domain.

6. The compound of claim 2, wherein the TfR1 binding domain and the albumin binding domain do not form a dimer.

7. The compound of claim 1, wherein the TfR1 binding domain is a peptide, a protein, an antibody, a Fc region, a Fab region, V_{H}H, or combinations thereof.

8. The compound of claim 7, wherein the TfR1 binding domain is a Fab region.

9. The compound of claim 3, wherein the first linker is attached to a heavy chain of the TfR1 binding domain or a light chain of the TfR1 binding domain.

10. The compound of claim 1, wherein the TfR1 binding domain has an affinity to a human TfR1 receptor of from about 1 nM to about 100 nM.

11. The compound of claim 1, wherein the TfR1 binding domain has an affinity to a human TfR1 receptor of from about 5 nM to less than 20 nM.

12. The compound of claim 1, wherein the TfR1 binding domain has an affinity to a human TfR1 receptor of about 10 nM.

13. The compound of claim 1, wherein the TfR1 binding domain comprises:
(a) a heavy chain, wherein the amino acid sequence of the heavy chain is SEQ ID NO. 15, SEQ ID NO. 16, SEQ ID NO. 17, or SEQ ID NO. 18, and
(b) a light chain, wherein the amino acid sequence of the light chain is SEQ ID NO. 19, SEQ ID NO. 20, SEQ ID NO. 21, or SEQ ID NO. 22.

14. The compound of claim 1, wherein the TfR1 binding domain comprises a heavy chain (HC) and a light chain (LC), wherein the amino acid sequence of the HC is SEQ ID NO. 15 and the amino acid sequence of the LC is SEQ ID NO. 19.

15. The compound of claim 2, wherein the albumin binding domain is a peptide, a protein, an antibody, a single domain antibody, a V_{H}H antibody fragment, a Fc region, a Fab region, or combinations thereof.

16. The compound of claim 2, wherein the albumin binding domain is a V_{H}H antibody fragment.

17. The compound of claim 3, wherein the albumin binding domain is attached to the C-terminus of the progranulin domain through L₂.

18. The compound of claim 2, wherein the amino acid sequence of the albumin binding domain is SEQ ID NO. 23 or SEQ ID NO. 24.

19. The compound of claim 3, wherein L₁ and L₂ are independently selected from a covalent bond, a peptide linker, a PEG linker, a disulfide bond, a thioacetal linkage, or a thioester linkage.

20. The compound of claim 3, wherein L₁ and L₂ are identical.

21. The compound of claim 3, wherein the amino acid sequence of L₁ and L₂ are SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, or SEQ ID NO. 14.

22. The compound of claim 3, wherein the amino acid sequence of L₁ and L₂ are SEQ ID NO. 7.

23. The compound of claim 1, wherein the progranulin domain is a naturally occurring sequence.

24. The compound of claim 1, wherein the progranulin domain comprises a fragment of progranulin, wherein the amino sequence of the fragment of progranulin is SEQ ID NO. 2.

25. The compound of any claim 1, wherein the amino acid sequence of the progranulin domain is SEQ ID NO. 1.

26. The compound of claim 1, wherein the compound comprises a heavy chain with an amino acid sequence having at least 90% sequence identity to the amino acid sequence given by SEQ ID NO. 25, SEQ ID NO. 26, or SEQ ID NO. 29.

27. The compound of claim 1, wherein the compound comprises a light chain with an amino acid sequence having at least 90% sequence identity to the amino acid sequence given by SEQ ID NO. 19, SEQ ID NO. 32, or SEQ ID NO. 33.

28. The compound of claim 1, wherein the compound comprises:
(a) a heavy chain, wherein the amino acid sequence of the heavy chain is SEQ ID NO. 25, SEQ ID NO. 26, or SEQ ID NO. 29; and
(b) a light chain, wherein the amino acid sequence of the light chain is SEQ ID NO. 19, SEQ ID NO. 32, or SEQ ID NO. 33.

29. The compound of claim 1, wherein the compound comprises a heavy chain (HC) and a light chain (LC), wherein the amino acid sequence of the HC is SEQ ID NO. 25 and the amino acid sequence of the LC is SEQ ID NO. 19.

30. The compound of claim 1, wherein the compound comprises a heavy chain (HC) and a light chain (LC), wherein the amino acid sequence of the HC is SEQ ID NO. 26 and the amino acid sequence of the LC is SEQ ID NO. 19.

31. The compound of claim 1, wherein the compound comprises a heavy chain (HC) and a light chain (LC), wherein the amino acid sequence of the HC is SEQ ID NO. 29 and the amino acid sequence of the LC is SEQ ID NO. 32.

32. The compound of claim 1, wherein the compound comprises a heavy chain (HC) and a light chain (LC), wherein the amino acid sequence of the HC is SEQ ID NO. 29 and the amino acid sequence of the LC is SEQ ID NO. 33.

33. A compound comprising an amino acid sequence having at least 90% sequence identity to the amino acid sequence given by SEQ ID NO. 25.

34. The compound of claim 33, wherein the compound comprises an amino acid sequence having at least 95% sequence identity to the amino acid sequence given by SEQ ID NO. 25.

35. The compound of claim 33, wherein the compound comprises a heavy chain (HC) and a light chain (LC), wherein the amino acid sequence of the HC is SEQ ID NO. 25 and the amino acid sequence of the LC is SEQ ID NO.

36. A compound comprising an amino acid sequence having at least 90% sequence identity to the amino acid sequence given by SEQ ID NO. 26.

37. The compound of claim 36, wherein the compound comprises an amino acid sequence having at least 95% sequence identity to the amino acid sequence given by SEQ ID NO. 26.

38. The compound of claim 36, wherein the compound comprises a heavy chain (HC) and a light chain (LC), wherein the amino acid sequence of the HC is SEQ ID NO. 26 and the amino acid sequence of the LC is SEQ ID NO. 19.

39. A method of treating a disorder, the method comprising administering the compound of claim 1 to a patient in need thereof.

40. The method of claim 39, wherein the disorder is neuronal ceroid lipofuscinosis type-11, frontotemporal dementia, Alzheimer's disease, Parkinson's disease, amyotrophic lateral sclerosis, or a combination thereof.

41. A composition comprising the compound of claim 1 and a pharmaceutically acceptable carrier.

42. A compound comprising a progranulin fragment, wherein the amino acid sequence of the progranulin fragment is SEQ ID NO. 2.

43. The compound of claim 42, wherein the progranulin fragment is from 100 residues to 500 residues in length.

44. The compound of claim 42, wherein the compound comprises:
(a) the progranulin fragment, and
(b) an TfR1 binding domain or an albumin binding domain.

45. The compound of claim 42, wherein the compound comprises:
(a) the progranulin fragment,
(b) an TfR1 binding domain, and
(c) an albumin binding domain.

46. The compound of claim 45, wherein the TfR1 binding domain is linked to the progranulin fragment with a first linker and the albumin binding domain is linked to the progranulin fragment or the TfR1 binding domain with a second linker.

47. The compound of claim 46, wherein the compound is of the formula:
Y-L₁-X-L₂-Z or Z-L₂-Y-L₁-X, and
wherein X is the progranulin fragment,
Y is the TfR1 binding domain,
Z is the albumin binding domain,
L₁ is the first linker, and
L₂ is the second linker.

48. The compound of claim 47, wherein the compound is of the formula:
Y-L₁-X-L₂-Z and
wherein the TfR1 binding domain is linked to the progranulin domain at an N-terminus of the progranulin domain and the albumin binding domain is linked to the progranulin at a C-terminus of the progranulin domain.

49. The compound of claim 47, wherein the TfR1 binding domain comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein
the amino acid sequence of HCDR1 is SEQ ID NO. 45,
the amino acid sequence of HCDR2 is SEQ ID NO. 46,
the amino acid sequence of HCDR3 is SEQ ID NO. 47,
the amino acid sequence of LCDR1 is SEQ ID NO. 48,
the amino acid sequence of LCDR2 is SEQ ID NO. 49, and
the amino acid sequence of LCDR3 is SEQ ID NO. 50.

50. The compound of claim 47, wherein the TfR1 binding domain and the albumin binding domain do not form a dimer.

51. The compound of claim 47, wherein the TfR1 binding domain is a peptide, a protein, an antibody, a Fc region, a Fab region, V_{H}H, or combinations thereof.

52. The compound of claim 47, wherein the TfR1 binding domain is a Fab region.

53. The compound of claim 47, wherein the first linker is attached to a heavy chain of the TfR1 binding domain or a light chain of the TfR1 binding domain.

54. The compound of claim 47, wherein the TfR1 binding domain has an affinity to a human TfR1 receptor of from about 1 nM to about 100 nM.

55. The compound of claim 47, wherein the TfR1 binding domain has an affinity to a human TfR1 receptor of from about 5 nM to less than 20 nM.

56. The compound of claim 47, wherein the TfR1 binding domain has an affinity to a human TfR1 receptor of about 10 nM.

57. The compound of claim 47, wherein the TfR1 binding domain comprises:
(a) a heavy chain, wherein the amino acid sequence of the heavy chain is SEQ ID NO. 15, SEQ ID NO. 16, SEQ ID NO. 17, or SEQ ID NO. 18, and
(b) a light chain, wherein the amino acid sequence of the light chain is SEQ ID NO. 19, SEQ ID NO. 20, SEQ ID NO. 21, or SEQ ID NO. 22.

58. The compound of claim 4, wherein the TfR1 binding domain comprises a heavy chain (HC) and a light chain (LC), wherein the amino acid sequence of the HC is SEQ ID NO. 15 and the amino acid sequence of the LC is SEQ ID NO.

59. The compound of claim 47, wherein the albumin binding domain the comprises complementarity determining regions (CDR) CDR1, CDR2, and CDR3, wherein
the amino acid sequence of CDR1 is SEQ ID NO: 51,
the amino acid sequence of CDR2 is SEQ ID NO: 52, and
the amino acid sequence of CDR3 is SEQ ID NO: 53.

60. The compound of claim 47, wherein the albumin binding domain is a peptide, a protein, an antibody, a single domain antibody, a V_{H}H antibody fragment, a Fc region, a Fab region, or combinations thereof.

61. The compound of claim 47, wherein the albumin binding domain is a V_{H}H antibody fragment.

62. The compound of claim 47, wherein the albumin binding domain is attached to the C-terminus of the progranulin domain through L₂.

63. The compound of claim 47, wherein the amino acid sequence of the albumin binding domain is SEQ ID NO. 23 or SEQ ID NO. 24.

64. The compound of claim 47, wherein L₁ and L₂ are independently selected from a covalent bond, a peptide linker, a PEG linker, a disulfide bond, a thioacetal linkage, or a thioester linkage.

65. The compound of claim 47, wherein L₁ and L₂ are identical.

66. The compound of claim 47, wherein the amino acid sequence of L₁ and L₂ are SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, or SEQ ID NO. 14.

67. The compound of claim 47, wherein the amino acid sequence of L₁ and L₂ are SEQ ID NO. 7.

68. The compound of claim 47, wherein the compound comprises a heavy chain with an amino acid sequence having at least 90% sequence identity to the amino acid sequence given by SEQ ID NO. 26 or SEQ ID NO. 29.

69. The compound of claim 47, wherein the compound comprises a light chain with an amino acid sequence having at least 90% sequence identity to the amino acid sequence given by SEQ ID NO. 19 or SEQ ID NO. 33.

70. The compound of claim 47, wherein the compound comprises:
(a) a heavy chain, wherein the amino acid sequence of the heavy chain is SEQ ID NO. 26 or SEQ ID NO. 29; and
(b) a light chain, wherein the amino acid sequence of the light chain is SEQ ID NO. 19 or SEQ ID NO. 33.

71. The compound of claim 47, wherein the compound comprises a heavy chain (HC) and a light chain (LC), wherein the amino acid sequence of the HC is SEQ ID NO. 26 and the amino acid sequence of the LC is SEQ ID NO. 19.

72. The compound of claim 47, wherein the compound comprises a heavy chain (HC) and a light chain (LC), wherein the amino acid sequence of the HC is SEQ ID NO. 29 and the amino acid sequence of the LC is SEQ ID NO. 33.

73. A method of treating a disorder, the method comprising administering the compound of claim 42 to a patient in need thereof.

74. The method of claim 73, wherein the disorder is neuronal ceroid lipofuscinosis type-11, frontotemporal dementia, Alzheimer's disease, Parkinson's disease, amyotrophic lateral sclerosis, or a combination thereof.

75. A composition comprising the compound of claim 42 and a pharmaceutically acceptable carrier.


SEQUENCE LISTING (73 sequences)

SEQ ID NO: 1 - protein, 576 aa, Homo sapiens
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 2 - protein, 414 aa, Homo sapiens
TPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 3 - protein, 573 aa, synthetic construct
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLQDPALR

SEQ ID NO: 4 - protein, 576 aa, synthetic construct
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRPIL

SEQ ID NO: 5 - protein, 5 aa, synthetic construct
GGGGQ

SEQ ID NO: 6 - protein, 10 aa, synthetic construct
GGGGQGGGGQ

SEQ ID NO: 7 - protein, 15 aa, synthetic construct
GGGGQGGGGQGGGGQ

SEQ ID NO: 8 - protein, 20 aa, synthetic construct
GGGGQGGGGQGGGGQGGGGQ

SEQ ID NO: 9 - protein, 25 aa, synthetic construct
GGGGQGGGGQGGGGQGGGGQGGGGQ

SEQ ID NO: 10 - protein, 5 aa, synthetic construct
GGGGS

SEQ ID NO: 11 - protein, 10 aa, synthetic construct
GGGGSGGGGS

SEQ ID NO: 12 - protein, 15 aa, synthetic construct
GGGGSGGGGSGGGGS

SEQ ID NO: 13 - protein, 20 aa, synthetic construct
GGGGSGGGGSGGGGSGGGGS

SEQ ID NO: 14 - protein, 25 aa, synthetic construct
GGGGSGGGGSGGGGSGGGGSGGGGS

SEQ ID NO: 15 - protein, 229 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 16 - protein, 229 aa, synthetic construct
EVQLVESGGGLVKPGGSLRLSCVASGFTFSSYSMNWVRQAPGKGLEWVSSISRSSSYIYYADSVKGRFTISRDNAKNSLYLQMNSLRAEDTAVYYCARIHGYSNSDAFDKWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 17 - protein, 226 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 18 - protein, 226 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 19 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 214 aa, synthetic construct
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 214 aa, synthetic construct
DIQMTQSPSAMSASVGDRVTITCRASQGISNYLAWFQQKPGKVPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 219 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - protein, 128 aa, synthetic construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 24 - protein, 128 aa, synthetic construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSPP

SEQ ID NO: 25 - protein, 963 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGQGGGGQGGGGQTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLLGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 26 - protein, 799 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGGGQGGGGQGGGGQTPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLLGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 27 - protein, 960 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGQGGGGQGGGGQTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLLGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 28 - protein, 963 aa, synthetic construct
EVQLVESGGGLVKPGGSLRLSCVASGFTFSSYSMNWVRQAPGKGLEWVSSISRSSSYIYYADSVKGRFTISRDNAKNSLYLQMNSLRAEDTAVYYCARIHGYSNSDAFDKWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGQGGGGQGGGGQTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLLGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 29 - protein, 380 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 30 - protein, 812 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGQGGGGQGGGGQGGGGQGGGGQTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLQDPALR

SEQ ID NO: 31 - protein, 650 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGQGGGGQGGGGQGGGGQGGGGQTPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALR

SEQ ID NO: 32 - protein, 815 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGQGGGGQGGGGQGGGGQGGGGQTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLQDPALRQLL

SEQ ID NO: 33 - protein, 653 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGQGGGGQGGGGQGGGGQGGGGQTPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 34 - protein, 379 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSPP

SEQ ID NO: 35 - protein, 820 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGQGGGGQGGGGQGGGGQGGGGQTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 36 - protein, 658 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGQGGGGQGGGGQGGGGQGGGGQTPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 37 - protein, 820 aa, synthetic construct
ALDMTQTASPVSAAVGGTVTINCQSSQSVYNNNWLAWYQQKPGQPPKLLIYDASTLASGVPSRFKGSGSGTQFTLTISGVQSDDSATYYCQGTYFSSGWSWAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGQGGGGQGGGGQGGGGQGGGGQTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 38 - protein, 820 aa, synthetic construct
AIDMTQTASPVSAAVGGTVTINCQSSQSVYNNNRLAWYQQKPGQPPKLLIYGASTLASGVPSRFKGSGSGTQFTLTISGVQCDDSATYYCQGTYFSSGWSWAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGQGGGGQGGGGQGGGGQGGGGQTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 39 - protein, 492 aa, synthetic construct
EVQLVESGGGLVQPGNSLTLSCVASGFTFSNYGMHWIRQAPKKCLEWIAMIYYDSSKMNYADTVKGRFTISRDNSKNTLYLEMNSLRSEDTAMYYCAVPTSHYVVDVWGQGVSVTVSSGGGGSGGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGCGTKLELKGGGGQGGGGQGGGGQDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 813 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 41 - protein, 212 aa, synthetic construct
AEVVMTQTPSSVSAAVGGTVTIKCQASQNINSWLSWYQQKPGQRPKLLIYSASTLASGVPSRFEGSGSGTEYTLTISDLECDDAATYYCQSSYGSSYDFGGGTEVVVKGDLVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 42 - protein, 446 aa, synthetic construct
QVQLVQSGAEVKKPGSSVKVSCTASGFSFSSGYWICWVRQAPGQGLEWMGCIHSVRSHMTYYAQKFQGRVTITADKSTSTAYMELSSLRSEDTAVYYCARDASGVWNYFTLWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 813 aa, synthetic construct
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLLGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 13 aa, synthetic construct
VASGFTFSSYSMN

SEQ ID NO: 46 - protein, 17 aa, synthetic construct
SISSSSSYIYYADSVKG

SEQ ID NO: 47 - protein, 14 aa, synthetic construct
ARRHGYSNSDAFDN

SEQ ID NO: 48 - protein, 11 aa, synthetic construct
RASQGISHYLV

SEQ ID NO: 49 - protein, 8 aa, synthetic construct
YAASSLQS

SEQ ID NO: 50 - protein, 9 aa, synthetic construct
LQHNSYPWT

SEQ ID NO: 51 - protein, 13 aa, synthetic construct
AASGRYIDETAVA

SEQ ID NO: 52 - protein, 17 aa, synthetic construct
GIGGGVDITYYADSVKG

SEQ ID NO: 53 - protein, 19 aa, synthetic construct
GARPGRPLITSKVADLYPY

SEQ ID NO: 54 - protein, 121 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSS

SEQ ID NO: 55 - protein, 609 aa, synthetic construct
HHHHHHGGGGQGGGGQGGGGQENLYFQSGGGGQTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 56 - protein, 10 aa, synthetic construct
SISSSSSYIY

SEQ ID NO: 57 - protein, 5 aa, synthetic construct
SYSMN

SEQ ID NO: 58 - protein, 12 aa, synthetic construct
RHGYSNSDAFDN

SEQ ID NO: 59 - protein, 11 aa, synthetic construct
RASQGISHYLV

SEQ ID NO: 60 - protein, 7 aa, synthetic construct
AASSLQS

SEQ ID NO: 61 - protein, 9 aa, synthetic construct
LQHNSYPWT

SEQ ID NO: 62 - protein, 8 aa, synthetic construct
GFTFSSYS

SEQ ID NO: 63 - protein, 8 aa, synthetic construct
ISSSSSYI

SEQ ID NO: 64 - protein, 14 aa, synthetic construct
ARRHGYSNSDAFDN

SEQ ID NO: 65 - protein, 6 aa, synthetic construct
QGISHY

SEQ ID NO: 66 - skipped

SEQ ID NO: 67 - protein, 9 aa, synthetic construct
LQHNSYPWT

SEQ ID NO: 68 - protein, 7 aa, synthetic construct
GFTFSSY

SEQ ID NO: 69 - protein, 6 aa, synthetic construct
SSSSSY

SEQ ID NO: 70 - protein, 12 aa, synthetic construct
RHGYSNSDAFDN

SEQ ID NO: 71 - protein, 11 aa, synthetic construct
RASQGISHYLV

SEQ ID NO: 72 - protein, 7 aa, synthetic construct
AASSLQS

SEQ ID NO: 73 - protein, 9 aa, synthetic construct
LQHNSYPWT